(19) United States
(12) Patent Application Publication
(10) Pub. No.: US 2008/0003220 A1
(43) Pub. Date: Jan. 3, 2008

(54) Buffering agents for biopharmaceutical formulations

(75) Inventor: Yatin R. Gokarn, Seattle, WA (US)

Correspondence Address: MCDERMOTT, WILL & EMERY, 4370 LA JOLLA VILLAGE DRIVE, SUITE 700, SAN DIEGO, CA, 92122, US

(73) Assignee: AMGEN, INC.

(21) Appl. No.: 11/788,770

(22) Filed: Apr. 19, 2007

Related U.S. Application Data
(60) Provisional application No. 60/794,201, filed on Apr. 21, 2006.
(60) Provisional application No. 60/876,726, filed on Dec. 21, 2006.

(51) Int. Cl. A61K 38/00; A61K 38/18; A61K 38/22; A61K 39/395; A61P 41/00

(52) U.S. Cl. 424/135.1; 424/130.1; 424/133.1; 514/2

(57) ABSTRACT

The invention provides a biopharmaceutical formulation including an aqueous solution having a propionate buffer with a pH from about 4.0 to about 6.0, at least one excipient and an effective amount of a therapeutic polypeptide. The propionate buffer can include a concentration selected from between about 1-50 mM, 2-30 mM, 3-20 mM, 4-10 mM and 5-8 mM. The therapeutic polypeptide included in a biopharmaceutical formulation of the invention can include an antibody, Fd, Fv, Fab, F(ab′), F(ab)₂, F(ab′)₂, single chain Fv (scFv), chimeric antibodies, diabodies, triabodies, tetrabodies, minibody, peptibody, hormone, growth factor or cell signaling molecule. The invention also provides a method of preparing a biopharmaceutical formulation. The method includes combining an aqueous solution having a propionate buffer with a pH from about 4.0 to about 6.0 and at least one excipient with an effective amount of a therapeutic polypeptide.

This application claims the benefit of priority of U.S. Provisional application Ser. No. 60/794,201, filed Apr. 21, 2006, and 60/876,726, filed Dec. 21, 2006, the entire contents of which are incorporated herein by reference.

### BACKGROUND OF THE INVENTION

This invention relates generally to medicines for the treatment of diseases and, more specifically to consistently stable formulations for biological molecule pharmaceuticals.

With the advent of recombinant DNA technology, protein-based therapeutics have become continually and increasingly commonplace in the repertoire of drugs available to medical practitioners for the treatment of a wide range of diseases from cancer to autoimmune diseases. Along with the scientific and technical advances that have occurred in the production of recombinant proteins, another reason for the success of protein therapeutics is their high specificity towards targets and their ability to exhibit superior safety profiles when compared to small molecule therapeutics. The ability to employ biological molecules as pharmaceuticals in the treatment of diseases has significantly advanced medical care and quality of life over the past quarter of a century. As of the year 2005, there were more than one hundred and fifty approved protein-based pharmaceuticals on the market and this number is expected to rise dramatically in the coming years. As with other pharmaceuticals, treatments with such pharmaceutical proteins require consistent and reproducible formulations to achieve safe and reliable therapeutic results.

Proteins known to exhibit various pharmacological actions in vivo are now capable of being produced in large amounts for various pharmaceutical applications. Long-term stability of a therapeutic protein is a particularly beneficial criterion for safe, consistent and efficacious treatments. Loss of functionality of the therapeutic within a preparation will decrease its effective concentration for a given administration. Similarly, undesired modifications of a therapeutic can affect the activity and/or the safety of a preparation, leading to loss of efficacy and risk of adverse side effects.

Proteins are complex molecules with defined primary, secondary, tertiary and in some cases quaternary structures, all of which play a role in imparting specific biological function. Structural complexity of biological pharmaceuticals such as proteins make them susceptible to various processes that result in structural and functional instability as well as loss of safety. With respect to these instability processes or degradation pathways, a protein can undergo a variety of covalent and non-covalent reactions or modifications in solution. For example, protein degradation pathways can be generally classified into two main categories: (i) physical degradation or non-covalent pathways, and (ii) chemical or covalent degradation pathways.

Protein drugs are susceptible to the physical degradation process of irreversible aggregation. Protein aggregation is of particular interest in biopharmaceutical production because it often results in diminished bioactivity that affects drug potency, and also can elicit serious immunological or antigenic reactions in patients. Chemical degradation of a protein therapeutic, including degradation of the chemical structure by, for example, chemical modification, also has been implicated in increasing its immunogenic potential. Thus, stable protein formulations require that both physical and chemical degradation pathways of the drug be minimized.

Proteins can degrade, for example, via physical processes such as interfacial adsorption and aggregation. Adsorption can significantly impact a protein drug's potency and stability. It can cause an appreciable loss in potency of low concentration dosage forms. A second consequence is that unfolding mediated adsorption at interfaces can often be an initiating step for irreversible aggregation in solution. In this respect, proteins tend to adsorb at liquid-solid, liquid-air, and liquid-liquid interfaces. Sufficient exposure of a protein's core at a hydrophobic surface can result in adsorption as a consequence of agitation, temperature or pH induced stresses. Further, proteins also are sensitive to, for example, pH, ionic strength, thermal, shear and interfacial stresses, all of which can lead to aggregation and result in instability.

Proteins also are subject to a variety of chemical modification and/or degradation reactions such as deamidation, isomerization, hydrolysis, disulfide scrambling, beta-elimination, oxidation and adduct formation. The principal hydrolytic mechanisms of degradation include peptide bond hydrolysis, deamidation of asparagine and glutamine and the isomerization of aspartic acid. A common feature of the hydrolytic degradation pathways is that one significant formulation variable, with respect to the rates of the reactions, is the solution pH.

For example, the hydrolysis of peptide bonds can be acid or base catalyzed. Asparagine and glutamine deamidation also are acid catalyzed below a pH of about 4. Asparagine deamidation at neutral pH occurs through a succinimidyl intermediate that is base catalyzed. The isomerization and racemization of aspartic acid residues can be rapid in slightly acidic to neutral pH (pH 4-8). In addition to the generalized pH effects, buffer salts and other excipients can affect the rates of the hydrolytic reactions.

Other exemplary degradation pathways include beta-elimination reactions, which can occur under alkaline pH conditions and lead to racemization or loss of part of the side-chain for certain amino acids. Oxidations of methionine, cysteine, histidine, tyrosine and tryptophan residues are exemplary covalent degradation pathways for proteins.

Because of the number and diversity of different reactions that can result in protein instability the composition of components in a biopharmaceutical formulation can significantly affect the extent of protein degradation and, consequently, the safety and efficacy of the therapeutic. The formulation of a biopharmaceutical also can affect the ease and frequency of administration and pain upon injection. For example, immunogenic reactions have not only been attributed to protein aggregates but also to mixed aggregates of the therapeutic protein with an inactive component contained in the formulation [Schellekens, H., Nat. Rev. Drug Discov. 1:457-62(2002); Hesmeling, et al., Pharm. Res. 22:1997-2006 (2005)].

However, despite the advances made in the utilization of proteins in therapeutic treatments and the knowledge of the instability process they can undergo, there is still a need to develop biopharmaceutical formulations with enhanced long-term stability characteristics. A biopharmaceutical formulation that retains long-term stability under a variety of conditions would provide an effective means of delivering an efficacious and safe amount of the biopharmaceutical. Retention of long-term stability in a biopharmaceutical formulation also would lower the production and treatment costs. Numerous recombinant or natural proteins could benefit from such consistently stable formulations and thereby provide more effective clinical results.

Thus, there exists a need for biopharmaceutical formulations that retain long-term stability under a variety of different manufacturing and storage conditions. The present invention satisfies this need and provides related advantages as well.

### SUMMARY OF THE INVENTION

The invention provides a biopharmaceutical formulation including an aqueous solution having a propionate buffer with a pH from about 4.0 to about 6.0, at least one excipient and an effective amount of a therapeutic polypeptide. The propionate buffer can include a concentration selected from between about 1-50 mM, 2-30 mM, 3-20 mM, 4-10 mM and 5-8 mM. The therapeutic polypeptide included in a biopharmaceutical formulation of the invention can include an antibody, Fd, Fv, Fab, F(ab′), F(ab)₂, F(ab′)₂, single chain Fv (scFv), chimeric antibodies, diabodies, triabodies, tetrabodies, minibody, peptibody, hormone, growth factor or cell signaling molecule. The invention also provides a method of preparing a biopharmaceutical formulation. The method includes combining an aqueous solution having a propionate buffer with a pH from about 4.0 to about 6.0 and at least one excipient with an effective amount of a therapeutic polypeptide.

### BRIEF DESCRIPTION OF THE DRAWINGS

FIG. 1 shows stability results under accelerated conditions as measured by size exclusion chromatography (SEC). The main peak is the intact polypeptide; HMW refers to high molecular weight fragments; LMW1 and LMW2 refer to low molecular weight fragments.

FIG. 2 shows SEC results for the pH profile of Emab (10 mg/mL), an IgG1 antibody, at 37 C for three weeks. The plot shows the dependence of the soluble HMW form (expressed as a percent of the polypeptide eluting bands derived from SEC) as a function of pH. The different buffer agents used in the study are listed.

FIG. 3 shows the dependence of the soluble LMW1 form (percent of eluted polypeptide) as a function of pH for the SEC results illustrated in FIG. 2. The different buffer agents used in the study are listed.

FIG. 4 shows the dependence of the soluble LMW2 form (percent of eluted polypeptide) as a function of pH for the SEC results illustrated in FIG. 2. The different buffer agents used in the study are listed.

FIG. 5 shows polypeptide stability in the presence of different excipients formulated in 20 mM sodium phosphate (pH 6). The amount of instability is indicated by the loss of main peak area as measured by SEC after incubation at 37 C for 11 weeks.

FIG. 6 shows HIAC subvisible particle measurements at a 6-month timepoint for three tested liquid formulations (A5S, PBS, and PASMT) at different temperatures. Data represent the average of three measurements and list the cumulative number of particles per mL.

FIG. 7 shows HIAC subvisible particle measurements representing the number of particles on a per dose basis for the same three formulations and temperature in FIG. 6. Horizontal lines drawn indicate the USP limits.

FIG. 8 shows SEC measurements summarizing polypeptide stability results related to dimer and fragmentation at designated temperature conditions for 6 months in designated candidate formulations (same as shown in FIGS. 6 and 7).

FIG. 9 shows the percent of LMW formation over time in three formulations (same shown in FIGS. 6, 7 and 8) maintained at 37 C.

FIG. 10 shows cation exchange chromatography (CEX) results for Emab incubated at the listed conditions for a six months. Accelerated data (green) depict significant changes affecting charge of the three eluting peaks. Eluting peaks correspond to 0 (about 20 min), 1 (about 22.5 min) and 2 (about 25 min) C-terminal lysine residues.

FIG. 11 shows a statistical representation of differing pH and EDTA conditions on dimer formation as measured by SEC.

FIG. 12 shows a statistical analysis illustrating a correlation between the change in dimer formation as measured by SEC and turbidity measured by absorbance at 400 nm.

FIG. 13 shows correlations between HIAC measurements and trends related to Tween-20, Tween-80, pH and EDTA shaken for 24 and 48 hours at room temperature.

FIG. 14 shows buffer capacity stability for biopharmaceutical formulations of the invention (labeled Pr5ST) compared to acetate buffered formulations (labeled A5ST).

FIG. 15 shows long-term stability of a therapeutic polypeptide at 37° C. in a biopharmaceutical formulation of the invention compared to acetate (designated by A) or succinate (designated by NaS) buffered formulations. Emab standard (unheated control).

FIG. 16 shows long-term stability of a therapeutic polypeptide at 4° C. in a biopharmaceutical formulation of the invention compared to acetate and succinate buffered formulations (designations the same as in FIG. 15).

### DETAILED DESCRIPTION OF THE INVENTION

This invention is directed to a biopharmaceutical formulation that exhibits optimal stabilizing capacity of polypeptides and other biopharmaceuticals. The biopharmaceutical formulation contains a propionic acid buffer system that is particularly useful in pH ranges between about 4.0-6.0. Biopharmaceuticals solubilized or included in a biopharmaceutical formulation of the invention exhibit stability for long periods of time, allowing the administration of a safe and effective amount of a therapeutic polypeptide or other biopharmaceutical. It is contemplated that propionic acid buffers also can provide other useful characteristics for biopharmaceutical formulations because they are less volatile than comparable buffer systems (e.g., acetate). Propionic acid also exhibits anti-microbial properties and therefore has inherent preservative qualities that can substitute for, or augment, one or more preservatives included in a biopharmaceutical formulation of the invention.

In one embodiment, the invention includes a biopharmaceutical formulation having a propionic acid buffer system. The weak acid component of the buffer system is supplied by sodium propionate to buffer the formulation and is present at a concentration of about 10 mM. In this specific embodiment, the biopharmaceutical formulation of the invention also contains about 5% sorbitol as an excipient and about 0.005% (w/v) polysorbate 20 as a surfactant. The final formulation is an aqueous solution that exhibits a pH of about 5.0 and maintains buffering capacity in the presence of a therapeutic polypeptide for at least 12-18 months.

As used herein, the term “biopharmaceutical” is intended to mean a macromolecule or biopolymer such as a polypeptide, nucleic acid, carbohydrate or lipid, or building block thereof, that is intended for use as a pharmaceutical. A “biopharmaceutical formulation” refers to a pharmaceutically acceptable medium that is compatible with a biopharmaceutical and is safe and non-toxic when administered to humans.

As used herein, the term “propionic acid” is intended to mean a liquid acid having the formula CH₃CH₂COOH. Propionic acid is soluble in water and alcohol with a melting point of −21 C and a boiling point of 141 C. A “propionic acid buffer” or “propionate buffer” as it is used herein is intended to refer to a buffer containing propionic acid in equilibrium with its conjugate base. A propionic acid buffer can provide optimal buffer capacity in the region of its pKₐ of 4.9, where buffer capacity refers to a resistance to change in pH when perturbed with either acid or base added to the solution. The propionic acid form of a propionic acid buffer of the invention can include, for example, propionic acid, propionate ion having the formula C₂H₅CO₂⁻ and/or propionate including propionic acid salt forms. A specific example of a propionic acid salt is sodium propionate, which has the formula (C₂H₅CO₂⁻) Na⁺. Other exemplary propionate salts that can be included in the buffer of the invention include, for example, potassium, calcium, organic amino or magnesium salt. Propionic acid and propionic acid buffers are well known by those skilled in the art.

As used herein, the term “excipient” is intended to mean a therapeutically inactive substance. Excipients can be included in a biopharmaceutical formulation for a wide variety of purposes including, for example, as a diluent, vehicle, buffer, stabilizer, tonicity agent, bulking agent, surfactant, cryoprotectant, lyoprotectant, anti-oxidant, metal ion source, chelating agent and/or preservative. Excipients include, for example, polyols such as sorbitol or mannitol; sugars such as sucrose, lactose or dextrose; polymers such as polyethylene glycol; salts such as NaCl, KCl or calcium phosphate, amino acids such as glycine, methionine or glutamic acid, surfactants, metal ions, buffer salts such as propionate, acetate or succinate, preservatives and polypeptides such as human serum albumin, as well as saline and water. Particularly useful excipients of the invention include sugars including sugar alcohols, reducing sugars, non-reducing sugars and sugar acids. Excipients are well known in the art and can be found described in, for example, Wang W., Int. J. Pharm. 185:129-88 (1999) and Wang W., Int. J. Pharm. 203:1-60 (2000).

Briefly, sugar alcohols, also known as a polyols, polyhydric alcohols, or polyalcohols, are hydrogenated forms of carbohydrate having a carbonyl group reduced to a primary or secondary hydroxyl group. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Specific examples of sugar alcohols include sorbitol, glycerol, mannitol, xylitol, maltitol, lactitol, erythritol and threitol.

Reducing sugars include, for example, sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose.

Non-reducing sugars contain an anomeric carbon that is an acetal and is not substantially reactive with amino acids or polypeptides to initiate a Maillard reaction. Sugars that reduce Fehling's solution or Tollen's reagent also are known as reducing sugars. Specific examples of non-reducing sugars include sucrose, trehalose, sorbose, sucralose, melezitose and raffinose.

Sugar acids include, for example, saccharic acids, gluconate and other polyhydroxy sugars and salts thereof.

Buffer excipients maintain the pH of liquid formulations through product shelf-life and maintain the pH of lyophilized formulations during the lyophilization process and upon reconstitution, for example.

Tonicity agents and/or stabilizers included in liquid formulations can be used, for example, to provide isotonicity, hypotonicity or hypertonicity to a formulation such that it is suitable for administration. Such excipients also can be used, for example, to facilitate maintenance of a biopharmaceuticals' structure and/or to minimize electrostatic, solution protein-protein interactions. Specific examples of tonicity agents and/or stabilizers include polyols, salts and/or amino acids. Tonicity agents and/or stabilizers included in lyophilized formulations can be used, for example, as a cryoprotectant to guard biopharmaceuticals from freezing stresses or as a lyoprotectant to stabilize biopharmaceuticals in the freeze-dried state. Specific examples of such cryo- and lyoprotectants include polyols, sugars and polymers.

Bulking agents are useful in lyophilized formulations to, for example, enhance product elegance and to prevent blowout. Bulking agents provide structural strength to the lyo cake and include, for example, mannitol and glycine.

Anti-oxidants are useful in liquid formulations to control protein oxidation and also can be used in lyophilized formulations to retard oxidation reactions.

Metal ions can be included in a liquid formulation, for example, as a co-factor and divalent cations such as zinc and magnesium can be utilized in suspension formulations. Chelating agents included in liquid formulations can be used, for example, to inhibit metal ion catalyzed reactions. With respect to lyophilized formulations, metal ions also can be included, for example, as a co-factor. Although chelating agents are generally omitted from lyophilized formulations, they also can be included as desired to reduce catalytic reactions during the lyophilization process and upon reconstitution.

Preservatives included in liquid and/or lyophilized formulations can be used, for example, to protect against microbial growth and are particularly beneficial in multi-dose formulations. In lyophilized formulations, preservatives are generally included in the reconstitution diluent. Benzyl alcohol is a specific example of a preservative useful in a formulation of the invention.

As used herein, the term “surfactant” is intended to mean a substance that functions to reduce the surface tension of a liquid in which it is dissolved. Surfactants can be included in a biopharmaceutical formulation for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants of the invention include non-ionic and ionic surfactants. Surfactants are well known in the art and can be found described in, for example, Randolph T. W. and Jones L. S., Surfactant-protein interactions. Pharm Biotechnol. 13:159-75 (2002).

Briefly, non-ionic surfactants include, for example, alkyl poly(ethylene oxide), alkyl polyglucosides such as octyl glucoside and decyl maltoside, fatty alcohols such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Polysorbate 20 is synonymous with TWEEN 20, sorbitan monolaurate and polyoxyethylenesorbitan monolaurate.

Ionic surfactants include, for example, anionic, cationic and zwitterionic surfactants. Anionic surfactants include, for example, sulfonate-based or carboxylate-based surfactants such as soaps, fatty acid salts, sodium dodecyl sulfate (SDS), ammonium lauryl sulfate and other alkyl sulfate salts. Cationic surfactants include, for example, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate.

As used herein, the term “therapeutic” when used in reference to a polypeptide of the invention is intended to mean that the polypeptide is intended for use in the cure, mitigation, treatment or prevention of disease in a human or other animal. Accordingly, a therapeutic polypeptide is a specific type of biopharmaceutical and can include a single polypeptide or two or more polypeptide subunits. A therapeutic polypeptide includes an antibody, a functional antibody fragment thereof, a peptibody or functional fragment thereof, growth factors, cytokines, cell signaling molecules and hormones. A wide variety of therapeutic polypeptides are well know in the art, all of which are included within the meaning of the term as it is used herein. Exemplary therapeutic polypeptides that can be used in an aqueous biopharmaceutical formulation of the invention include, for example, antibodies such as Epratuzumab® (Emab) and functional fragments to a wide variety of antigens, interleukins, G-CSF, GM-CSF, kinases, TNF and TNFR ligands, cyclins and erythropoietin.

As used herein, the term “effective amount” when used in reference to a therapeutic biopharmaceutical such as a therapeutic polypeptide is intended to mean an amount of the therapeutic molecule sufficient to ameliorate at least one symptom associated with a targeted disease or physiological condition.

The invention provides a biopharmaceutical formulation including an aqueous solution having a propionate buffer with a pH from about 4.0 to about 6.0, at least one excipient and an effective amount of a therapeutic polypeptide.

A biopharmaceutical formulation of the invention exhibits optimal properties for administration, storage and manipulation of biopharmaceuticals. Manipulation includes, for example, lyophilization, reconstitution, dilution, titration and the like. The aqueous buffering component of a formulation of the invention is efficient to prepare and can easily be combined with a desired biopharmaceutical using any of a variety of methods well know in the art, avoiding cumbersome and, sometimes lengthy, preparatory and/or intermediate steps. Additionally, the aqueous propionic acid buffering component is compatible with a wide variety of excipients and surfactants that facilitate stability of a biopharmaceutical. These and other attributes of a biopharmaceutical formulation of the invention described herein allow stable formulations of bioactive molecules to be prepared and maintained over periods exceeding 12-18 months or more.

Stability of a biopharmaceutical formulation of the invention refers to the retention of structure and/or function of a biopharmaceutical within a formulation. A biopharmaceutical in a formulation of the invention will exhibit attributes such as resistance to change or deterioration that affect stability or function and therefore maintain consistent functional characteristics over time. Accordingly, biopharmaceutical formulations of the invention will exhibit, for example, reliability and safety with respect to activity per volume or activity units.

In one embodiment, the stability of a biopharmaceutical within a formulation of the invention includes, for example, the retention of physical and/or chemical stability. Biopharmaceutical stability can be assessed by, for example, determining whether the biopharmaceutical has been subjected to a physical degradation and/or chemical degradation pathway such as those described previously, including chemical modification of its structure. Retention in stability of a biopharmaceutical in a formulation of the invention includes, for example, retention of physical or chemical stability between about 80-100%, 85-99%, 90-98%, 92-96% or 94-95% compared to the stability of the biopharmaceutical at an initial time point. Accordingly, stability of a biopharmaceutical within a formulation of the invention includes retention of stability greater than 99.5%, at least about 99%, 98%, 97%, 96%, 95%, 94%, 93%, 92%, 91%, 90%, 89%, 88%, 87%, 86%, 85%, 84%, 83%, 82%, 81% or 80% compared to the stability of the biopharmaceutical at an initial time point.

In a further embodiment, stability of a biopharmaceutical within a formulation of the invention includes, for example, retention of activity. Biopharmaceutical activity can be assessed using, for example, an in vitro, in vivo and/or in situ assay indicative of the biopharmaceutical's function. Retention of stability of a biopharmaceutical in a formulation of the invention includes, for example, retention of activity between about 50-100% or more, depending on the variability of the assay. For example, retention in stability can include retention of activity between about 60-90% or 70-80% compared to the activity of the biopharmaceutical at an initial time point. Accordingly, stability of a biopharmaceutical within a formulation of the invention includes retention of activity of at least about 50%, 55%, 60%, 65%, 70%, 75%, 80%, 85%, 90%, 95% or 100% and can include activity measurements greater than 100% such as 105%, 110%, 115%, 120%, 125% or 150% or more compared to the activity of the biopharmaceutical at an initial time point. Generally, an initial time point is selected to be the time that a biopharmaceutical is first prepared in a biopharmaceutical formulation of the invention or first examined for quality (i.e., meets release specifications). An initial time point also can include the time at which a biopharmaceutical is reformulated in a biopharmaceutical formulation of the invention. The reformulation can be, for example, at a higher concentration, lower concentration or at the same concentration of an initial preparation.

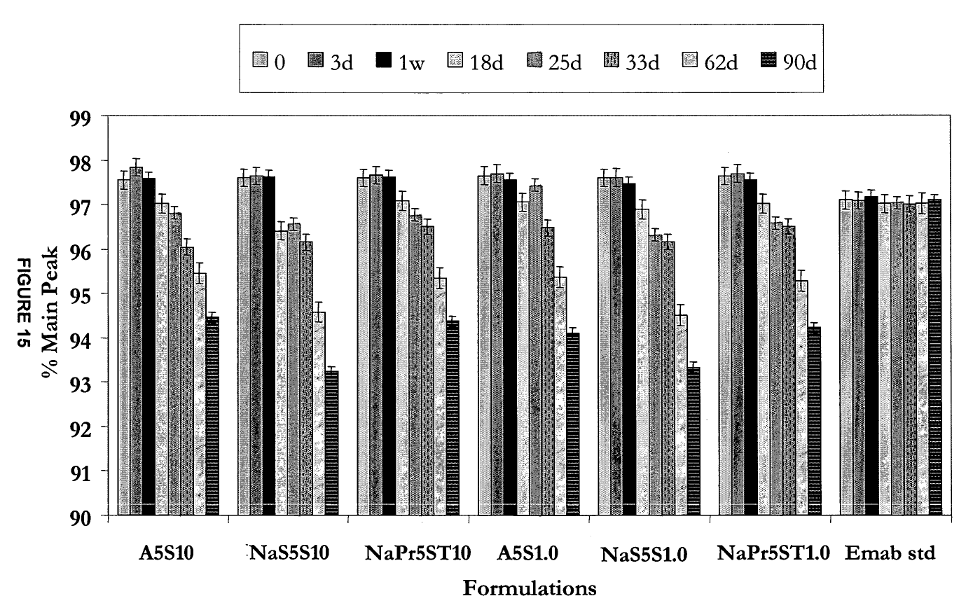

The stability of a biopharmaceutical in a biopharmaceutical formulation of the invention is particularly retained at temperatures above 4° C. such as at room temperature, about 23° C., or higher, including 37° C. This greater retention in stability at higher temperatures is shown by the greater retention of the main peak of the propionic acid buffer biopharmaceutical formulations of the invention shown in FIG. 15 compared to certain other buffers, particularly with the higher concentrations of the referenced biopharmaceutical.

A biopharmaceutical formulation of the invention can be prepared to be isotonic with a reference solution or fluid (i.e., blood serum). An isotonic solution has a substantially similar amount of dissolved solute in it compared to the things around it so that it is osmotically stable. Unless expressly compared to a specific solution or fluid, isotonic or isotonicity is exemplary used herein by reference to human blood serum (e.g., 300 mOsmol/kg). Therefore, an isotonic biopharmaceutical formulation of the invention will contain a substantially similar concentration of solutes or exhibit substantially similar osmotic pressure as human blood. In general, an isotonic solution contains about the same concentration of solutes as normal saline for humans and many other mammals, which is about 0.9 weight percent (0.009 g/ml) salt in aqueous solution (e.g., 0.009 g/ml NaCl). Biopharmaceutical formulations of the invention also can include hypotonic or hypertonic solution preparations.

A biopharmaceutical formulation can be prepared in any of a variety of ways well known in the art to produce a propionate buffer component having a desired pH, at least one excipient and an effective amount of a biopharmaceutical. In this regard, the buffering capacity of a biopharmaceutical formulation of the invention is supplied by the weak acid propionic acid, which exhibits a strong buffering capacity at a pH range that is within about 1 pH unit of its pKₐ. Propionic acid has a pKₐ of 4.9 which is optimal for many biological molecules including, for example, macromolecules having important biochemical and structural functions.

The propionic acid component can be supplied to the buffering system in a variety of different propionic acid forms. For example, the propionic acid component can be supplied as propionic acid, propionate salt or any other form that is available or that can be produced using chemical synthesis. Propionate in its salt form is particularly useful for producing a propionate buffering system of a biopharmaceutical formulation because it is commercially available in highly purified form. Propionate salts include, for example, those described previously as well as others known in the art. A highly purified form of a biopharmaceutical formulation component refers to pharmaceutical grade purity level, which is sufficiently pure to administer to a human such that it is devoid of contaminants so as to be safe and non-toxic.

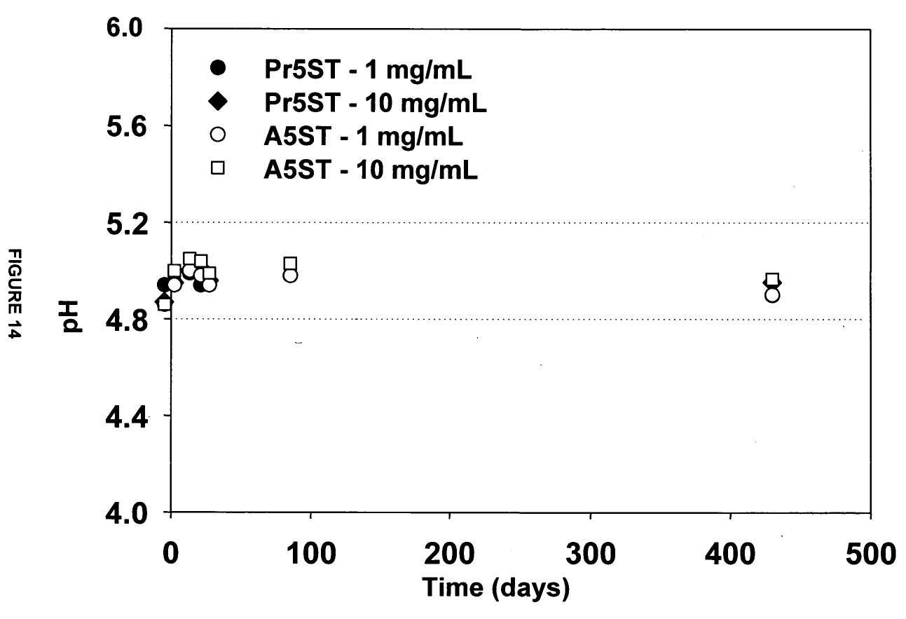

Propionic acid and propionate buffers are well known in by those skilled in the art. A biopharmaceutical formulation of the invention will contain a concentration of, for example, propionic acid or propionate having sufficient buffering capacity to maintain a selected pH of a formulation at a selected temperature. Useful concentrations of propionic acid or propionate, for example, include between about 1-150 mM and as high as 200 mM or more. For example, in some instances, it can be desirable to include up to 1 M propionic acid or propionate to produce a hypertonic formulation of the invention. Such hypertonic solutions can be diluted to produce an isotonic formulation prior to use if desired. By way of exemplification, useful concentrations of propionic acid or propionate include, for example, between about 1-200 mM, 5-175 mM, 10-150 mM, 15-125 mM, 20-100 mM, 25-80 mM, 30-75 mM, 35-70 mM, 40-65 mM and 45-60 mM. Other useful concentrations of propionic acid or propionate include, for example, between about 1-50 mM, 2-30 mM, 3-20 mM, 4-10 mM and 5-8 mM. Accordingly, a propionic acid or propionate concentration of about 1, 2, 3, 4, 5, 6, 7, 8, 9, 10, 11, 12, 13, 14, 15, 16, 17, 18, 19 or 20 mM or more. All values above and below these exemplary concentrations also can be used in a biopharmaceutical formulation. Therefore, a biopharmaceutical formulation of the invention can have a propionic acid or propionate less than 1 mM or greater than 20 mM including, for example, 21, 22, 23, 24, 25, 30, 35, 40, 45 or 50 mM or more propionic acid or propionate. A biopharmaceutical formulation is exemplified in the Examples below and shown in FIGS. 14-16 containing a propionate concentration of about 10 mM.

As described previously, the pKₐ of a propionic acid buffer in a biopharmaceutical formulation of the invention is particularly suitable for use with biopharmaceuticals because it has a strong buffering capacity between about pH 4-6, which can be optimal for maintenance of biopharmaceutical stability. A propionic acid buffer component of a biopharmaceutical formulation of the invention can be prepared to exhibit any effective buffering capacity within a pH range of between about 4.0 to 6.0. Exemplary pH ranges of a propionic acid buffer and/or the final biopharmaceutical formulation can include pH ranges between about 3.5-6.5, between about 4.0-6.0, between about 4.5-5.5, between about 4.8-5.2 or about 5.0. Accordingly, a propionic acid buffer and/or the final biopharmaceutical formulation can be prepared to have a pH of about 3.0 or less, about 3.5, 4.0, 4.5, 4.8, 5.0, 5.2, 5.5, 6.0, 6.5 or about 7.0 or more. All pH values above, below and in between these exemplary values also can be used in a propionic acid buffer and/or the final biopharmaceutical formulation. Therefore, for example, a propionic acid buffer component and/or the final biopharmaceutical formulation can be prepared to have a pH less than 3.5, greater than 6.5 and all values within these ranges. Those skilled in the art will understand that much of the strength of the buffering capacity of a buffer will decrease outside of about 1 pH unit of its pKₐ and, given the teachings and guidance provided herein, can determine whether inclusion of a propionic acid buffer below a pH of about 3.5 or above a pH of about 6.5 is useful in a biopharmaceutical formulation of the invention.

A propionic acid buffer component of a biopharmaceutical formulation of the invention can include one or more excipients. As described previously, one role of an included excipient is to provide stabilization of the biopharmaceutical against stresses that can occur during manufacturing, shipping and storage. To accomplish this role, at least one excipient can function as a buffer, stabilizer, tonicity agent, bulking agent, surfactant, cryoprotectant, lyoprotectant, anti-oxidant, metal ion source, chelating agent and/or preservative. In addition, at least one excipient also can function as a diluent and/or vehicle or be employed to reduce viscosity in high concentration biopharmaceutical formulations in order to enable their delivery and/or enhance patient convenience.

Similarly, at least one excipient additionally can confer more than one of the above functions onto a formulation of the invention. Alternatively, two or more excipients can be included in a biopharmaceutical formulation of the invention to perform more than one of the above or other functions. For example, an excipient can be included as a component in a biopharmaceutical formulation of the invention to change, adjust or optimize the osmolality of the formulation, thereby acting as a tonicifier. Similarly, a tonicity agent and a surfactant can both be included in a biopharmaceutical formulation of the invention to both adjust the osmolality and control aggregation. Excipients, their use, formulation and characteristics are well known in the art and can be found described in, for example, Wang W., Int. J. Pharm. 185:129-88 (1999) and Wang W., Int. J. Pharm. 203:1-60 (2000).

In general, excipients can be chosen on the basis of the mechanisms by which they stabilize proteins against various chemical and physical stresses. As described herein, certain excipients are beneficial to include so as to alleviate the effects of a specific stress or to regulate a particular susceptibility of a specific biopharmaceutical. Other excipients are beneficial to include because they have more general effects on the physical and covalent stabilities of proteins. Particularly useful excipients include those chemically and functionally innocuous or compatible with aqueous buffer solutions and biopharmaceuticals so as to optimize the stability properties of a formulation. Various such excipients are described herein as exemplary excipients exhibiting chemical compatibility with the aqueous biopharmaceutical formulations of the invention and functional compatibility with the biopharmaceutical included in such formulations. Those skilled in the art will understand that the teachings and guidance provided herein with respect to the exemplified excipients are equally applicable to the use of a wide range of other excipients well known in the art.

For example, optimal excipients chosen to enhance or confer stability of a biopharmaceutical within a formulation include those that are substantially free from reacting with functional groups on the biopharmaceutical. In this regard, both reducing and non-reducing sugars can be used as an excipient in a biopharmaceutical formulation of the invention. However, because reducing sugars contain a hemiacetal group they can react and form adducts or other modifications with amino groups on amino acid side chains of polypeptides (i.e., glycosylation). Similarly, excipients such as citrate, succinate or histidine also can form adducts with amino acid side chains. Given the teachings and guidance provided herein, those skilled in the art will known that greater retention of stability for a given polypeptide biopharmaceutical can be achieved by choosing a non-reducing sugar over a reducing sugar or over other amino acid-reactive excipients such as those exemplified above.

Optimal excipients also are chosen to enhance or provide stabilization with reference to the mode of administration for an aqueous biopharmaceutical formulation of the invention. For example, parenteral routes of intravenous (IV), subcutaneous (SC) or intramuscular (IM) administration can be more safe and efficacious when all components of the formulation maintain physical and chemical stability during manufacture, storage and administration. Those skilled in the art will know to employ one or more excipients that maintain maximal stability of the active form of a biopharmaceutical given, for example, a particular manufacturing or storage condition or a particular mode of administration. The excipients exemplified herein for use in a biopharmaceutical formulation exhibit these and other characteristics.

The amount or concentration of excipient to use in a biopharmaceutical formulation of the invention will vary depending on, for example, the amount of biopharmaceutical included in the formulation, the amount of other excipients included in the desired formulation, whether a diluent is desired or needed, the amount or volume of other components of the formulation, the total amount of components within a formulation, the specific activity of the biopharmaceutical and the desired tonicity or osmolality to be achieved. Specific examples for excipient concentrations are exemplified further below. Further, different types of excipients can be combined into a single biopharmaceutical formulation. Accordingly, a biopharmaceutical formulation of the invention can contain a single excipient, two, three or four or more different types of excipients. Combinations of excipients can be particularly useful in conjunction with a biopharmaceutical formulation that contains two or more different biopharmaceuticals. The excipients can exhibit similar or different chemical properties.

Given the teachings and guidance provided herein, those skilled in the art will know what amount or range of excipient can be included in any particular formulation to achieve a biopharmaceutical formulation of the invention that promotes retention in stability of the biopharmaceutical. For example, the amount and type of a salt to be included in a biopharmaceutical formulation of the invention can be selected based on to the desired osmolality (i.e., isotonic, hypotonic or hypertonic) of the final solution as well as the amounts and osmolality of other components to be included in the formulation. Similarly, by exemplification with reference to the type of polyol or sugar included in a formulation, the amount of such an excipient will depend on its osmolality. Inclusion of about 5% sorbitol can achieve isotonicity while about 9% of a sucrose excipient is needed to achieve isotonicity. Selection of the amount or range of concentrations of one or more excipients that can be included within a biopharmaceutical formulation of the invention has been exemplified above by reference to salts, polyols and sugars. However, those skilled in the art will understand that the considerations described herein and further exemplified by reference to specific excipients are equally applicable to all types and combinations of excipients including, for example, salts, amino acids, other tonicity agents, surfactants, stabilizers, bulking agents, cryoprotectants, lyoprotectants, anti-oxidants, metal ions, chelating agents and/or preservatives.

Excipients can be included in a biopharmaceutical formulation of the invention at concentration ranges generally between about 1-40% (w/v), between about 5-35% (w/v), between about 10-30% (w/v), between about 15-25% (w/v) or about 20% (w/v). Concentrations as high as about 45% (w/v), 50% (w/v) or more than 50% (w/v) in certain instances also can be employed in the biopharmaceutical formulations of the invention. For example, in some instances, it can be desirable to include concentrations up to 60% (w/v) or 75% (w/v) to produce a hypertonic formulation of the invention. Such hypertonic solutions can be diluted to produce an isotonic formulation prior to use if desired. Other useful concentration ranges include between about 1-20%, particularly between about 2-18% (w/v), more particularly between about 4-16% (w/v), even more particularly between about 6-14% (w/v) or between about 8-12% (w/v) or about 10% (w/v). Excipient concentrations and/or amounts less than, greater than or in between these ranges also can be used in a biopharmaceutical formulation of the invention. For example, one or more excipients can be included in a biopharmaceutical formulation which constitute less than about 1% (w/v). Similarly, a biopharmaceutical formulation can contain a concentration of one or more excipients greater than about 40% (w/v). Accordingly, a biopharmaceutical formulation of the invention can be produced that contains essentially any desired concentration or amount of one or more excipients including, for example, 1, 2, 3, 4, 5, 6, 7, 8, 9, 10, 11, 12, 13, 14, 15, 16, 17, 18, 19 or 20% (w/v) or more. An example is provided below for a biopharmaceutical formulation of a polypeptide having about 10.0% excipient.

Various excipients useful in a biopharmaceutical formulation of the invention have been described previously. In the specific biopharmaceutical formulation described in Example II, one exemplified excipient is sorbitol, which is employed as a tonicity agent and/or stabilizer. Another excipient exemplified in the biopharmaceutical formulation described in Example II is polysorbate 20, which is employed in that specific formulation as a surfactant. Other excipients useful in either a liquid or lyophilized biopharmaceutical formulation of the invention include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol and phenol. Excipients such as these as well as others known in the art can be found described in, for example, Wang W., supra, (1999) and Wang W., supra, (2000).

A propionic acid buffer component of a biopharmaceutical formulation of the invention also can include one or more surfactants as an excipient. As described previously, one role of surfactants in a formulation of the invention is to prevent or minimize aggregation and/or adsorption such as surface-induced degradation. At sufficient concentrations, generally about the surfactant's critical micellar concentration, a surface layer of surfactant molecules serve to prevent protein molecules from adsorbing at the interface. Thereby, surface-induced degradation is minimized. Surfactant, their use, formulation and characteristics for biopharmaceutical formulations are well known in the art and can be found described in, for example, Randolph and Jones, supra, (2002).

Optimal surfactants to include in a biopharmaceutical formulation of the invention can be chosen, for example, to enhance or promote retention in stability of the biopharmaceutical by preventing or reducing aggregation and/or adsorption. For example, sorbitan fatty acid esters such as the polysorbates are surfactants exhibiting with a wide range of hydrophilic and emulsifying characteristics. They can be used individually or in combination with other surfactants to cover a wide range of stabilization needs. Such characteristics are particularly suitable for use with biopharmaceuticals because they can be tailored to cover the wide range of hydrophobic and hydrophilic characteristics of biopharmaceuticals. Considerations for selecting a surfactant include those described previously with reference to excipients in general as well as the hydrophobic character and critical micellar concentration of the surfactant. The surfactants exemplified herein, as well as many others well known in the art can be used in a biopharmaceutical formulation of the invention.

Surfactant concentration ranges for a biopharmaceutical formulation of the invention include those described previously with reference to excipients in general with particularly useful concentrations being less than about 1% (w/v). In this regard, surfactant concentrations generally can be used at ranges between about 0.001-0.10% (w/v), particularly between about 0.002-0.05% (w/v), more particularly between about 0.003-0.01% (w/v), even more particularly between about 0.004-0.008% (w/v) or between about 0.005-0.006% (w/v). Surfactant concentrations and/or amounts less than, greater than or in between these ranges also can be used in a biopharmaceutical formulation of the invention. For example, one or more surfactants can be included in a biopharmaceutical formulation which constitute less than about 0.001% (w/v). Similarly, a biopharmaceutical formulation can contain a concentration of one or more surfactants greater than about 0.10% (w/v). Accordingly, a biopharmaceutical formulation of the invention can be produced that contains essentially any desired concentration or amount of one or more surfactants including, for example, 0.001, 0.002, 0.003, 0.004, 0.005, 0.006, 0.007, 0.008, 0.009, 0.010, 0.02, 0.03, 0.04, 0.05, 0.06, 0.07, 0.08, 0.09 or 0.10% (w/v) or more.

Various surfactants useful as an excipient in a biopharmaceutical formulation of the invention have been described previously. Other surfactants useful in either a liquid or lyophilized biopharmaceutical formulation of the invention include, for example, sugar esters such as esters lauric acid (C12), palmitic acid (C16), stearic acid (C18), macrogol cetostearyl ethers, macrogol lauryl ethers, macrogol oleyl ether, macrogol oleate, macrogol stearate, macrogol glycerol ricinoleate, macrogol glycerol hydroxystearate; alkyl polyglucosides such as octyl glucoside and decyl maltoside; fatty alcohols such as cetyl alcohol and oleyl alcohol, and cocamides such as cocamide MEA, DEA, TEA, other non-ionic surfactants and other ionic surfactants.

Therefore, the invention provides a biopharmaceutical formulation that includes an aqueous solution having between about 1-100 mM propionate with a pH from about 4.0 to about 6.0, polyol between about 1-10%, polysorbate 20 between about 0.001-0.010% and an effective amount of a therapeutic polypeptide. The biopharmaceutical formulation of the invention also can include about 10 mM of sodium propionate having a pH of about 5.0, about 5% sorbitol and about 0.005% polysorbate 20. Various other formulation components, combinations of components and concentrations thereof also can be included in a biopharmaceutical formulation of the invention.

Further provided is a biopharmaceutical formulation having a therapeutic polypeptide as the biopharmaceutical component of the formulation. The therapeutic polypeptide includes an antibody, a functional fragment of an antibody, a peptibody, a hormone, a growth factor or a cell signaling molecule.

Also included within a biopharmaceutical formulation of the invention is a biopharmaceutical. A biopharmaceutical of the invention includes, for example, a macromolecule or biopolymer such as a polypeptide, nucleic acid, lipid, carbohydrate employed as an active pharmaceutical ingredient or building block thereof, that can be used in the diagnosis, treatment or prevention of a pathological condition or as a component of a medication. For example, the biopharmaceutical formulations of the invention are applicable to, and facilitate retention in stability for, polypeptides, glycopolypeptides, peptidoglycans, DNA such as genomic DNA, cDNA and the like, RNA such as mRNA, RNAi, SNRPS, and the like, carbohydrates contemplated as an active pharmaceutical ingredient which can include monosaccharides, polysaccharides, N-linked sugars, O-linked sugars, leptins and the like, lipids such as phospholipids, glycolipids, fatty acids, polyamines, isoprenoids, amino acids, nucleotides, neurotransmitters and co-factors, as well as many other macromolecules, biopolymers and building blocks thereof, endogenous to mammalian physiological systems, including human. These and other biopharmaceuticals are well known to those skilled in the art and can be included in a biopharmaceutical formulation of the invention for use in the diagnosis, treatment or prevention of a pathological condition or as a component of a medication.

Given the teachings and guidance provided herein, those skilled in the art will understand that a biopharmaceutical formulation of the invention is equally applicable to all types of biopharmaceuticals, including those exemplified above as well as others well known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, as described previously, non-reducing sugars exhibit favorable excipient properties when used with polypeptide biopharmaceuticals compared to reducing sugars. Accordingly, the biopharmaceutical formulations of the invention are exemplified further below with reference to polypeptide biopharmaceuticals. However, the range of applicability, chemical and physical properties, considerations and methodology applied to polypeptide biopharmaceutical are similarly applicable to biopharmaceuticals other than polypeptide biopharmaceuticals.

Exemplary types of polypeptide biopharmaceuticals applicable for use in a biopharmaceutical formulation of the invention include all types of therapeutic polypeptides including, for example, the immunoglobulin superfamily of polypeptides, growth factors, cytokines, cell signaling molecules and hormones. Exemplary polypeptide biopharmaceuticals applicable for use in a biopharmaceutical formulation of the invention include all therapeutic polypeptides including, for example, antibodies and functional fragments thereof, interleukins, G-CSF, GM-CSF, kinases, TNF and TNFR ligands including Fhm, cyclins, erythropoietin, nerve growth factors (NGF), developmentally regulated nerve growth factor VGF, neurotrophic factors, neurotrophic factor NNT-1, Eph receptor, Eph receptor ligands; Eph-like receptor, Eph-like receptor ligands, inhibitors of apoptosis proteins (IAP), Thy-1 specific protein, Hek ligand (hek-L), Elk receptor and Elk receptor ligands, STATs, collagenase inhibitor, osteoprotegerin (OPG), APRIL/G70, AGP-3/BLYS, BCMA, TACI, Her-2/neu, Apolipoprotein polyeptides, integrins, tissue inhibitor of metalloproteinases, C3b/C4b complement receptor, SHC binding protein, DKR polypeptides, extracellular matrix polypeptides, antibodies to the above therapeutic polypeptides and antibody functional fragments thereof, antibodies to receptors for the above therapeutic polypeptides and antibody functional fragments thereof, functional polypeptide fragments thereof, fusion polypeptides, chimeric polypeptides and the like.

Specific examples of commercially available biopharmaceuticals applicable for use in a biopharmaceutical formulation of the invention include, for example, ENBREL (Etanercept; a CHO expressed dimeric fusion protein ((Amgen, Inc.)); EPOGEN (Epoetin alfa; a mammalian cell expressed glycoprotein (Amgen, Inc.)); INFERGEN® (Interferon alfacon-1; an E. Coli expressed recombinant protein (Amgen, Inc.)); KINERET® (anakinra; an E. coli expressed recombinant, nonglycosylated form of the human interleukin-1 receptor antagonist (IL-1Ra) (Amgen, Inc.)); ARANESP (darbepoetin alfa; a CHO expressed recombinant human erythropoiesis stimulating protein (Amgen, Inc.)); NEULASTA (pegfilgrastim; covalent conjugate of recombinant methionyl human G-CSF and 20 kD PEG (Amgen, Inc.)); NEUPOGEN (Filgrastim; an E. coli expressed human granulocyte colony-stimulating factor (G-CSF) (Amgen, Inc.)), and STEMGEN (Ancestim, stem cell factor; an E. Coli expressed recombinant human protein (Amgen, Inc.)). These and all other commercially available biopharmaceuticals can be, for example, reformulated in a biopharmaceutical formulation of the invention at the time of production, prior to use and/or prior to short or long term storage.

By further illustration of the range of biopharmaceutical applicability of a biopharmaceutical formulation of the invention, described further below are exemplary types of antibodies and functional fragments thereof, that can be employed as a therapeutic polypeptide in a biopharmaceutical formulation of the invention. As described previously, the chemical and physical properties, formulation considerations and methodology applicable to antibodies and functional fragments thereof, are similarly applicable to biopharmaceuticals including other polypeptide biopharmaceuticals.

An antibody or immunoglobulin is a polypeptide that has specific affinity for a molecular target or antigen. The term refers to a polypeptide product of B cells within the immunoglobulin class of polypeptides which is composed of heavy and light chain. A monoclonal antibody refers to an antibody that is the product of a single cell clone or hybridoma. Monoclonal antibody also refers to an antibody produced by recombinant methods from heavy and light chain encoding immunoglobulin genes to produce a single molecular immunoglobulin species. Amino acid sequences for antibodies within a monoclonal antibody preparation are substantially homogeneous and the binding activity of antibodies within such a preparation exhibit substantially the same antigen binding activity when compared in the same or similar binding assay. As described further below, antibody and monoclonal antibody characteristics are well known in the art.

Monoclonal antibodies can be prepared using a wide variety of methods known in the art including the use of hybridoma, recombinant, myeloma cell-line expressed, phage display and combinatorial antibody library methodologies, or a combination thereof. For example, monoclonal antibodies can be produced using hybridoma techniques including those known in the art and taught, for example, in Harlow and Lane., Antibodies: A Laboratory Manual, Cold Spring Harbor Laboratory Press (1989); Hammerling, et al., in: Monoclonal Antibodies and T-Cell Hybridomas 563-681, Elsevier, N.Y. (198 1); Harlow et al., Using Antibodies: A Laboratory Manual, Cold Spring Harbor Laboratory Press (1999), and Antibody Engineering. A Practical Guide, C. A. K. Borrebaeck, Ed., W.H. Freeman and Co., Publishers, New York, pp. 103-120 (1991). Examples of known methods for producing monoclonal antibodies by recombinant, phage display and combinatorial antibody library methods, including libraries derived from immunized and naive animals can be found described in Antibody Engineering: A Practical Guide, C. A. K. Borrebaeck, Ed., supra. A monoclonal antibody for use as a biopharmaceutical is not limited to antibodies produced through hybridoma technology. Rather, as described previously, a monoclonal antibody refers to an antibody that is derived from a single clone, including any eukaryotic, prokaryotic, or phage clone, and not the method by which it is produced.

An antibody functional fragment refers to a portion of an antibody which retains some or all of its target-specific binding activity. Such functional fragments can include, for example, antibody functional fragments such as Fd, Fv, Fab, F(ab′), F(ab)₂, F(ab′)₂, single chain Fv (scFv), chimeric antibodies, diabodies, triabodies, tetrabodies and minibody. Other functional fragments can include, for example, heavy (H) or light (L) chain polypeptides, variable heavy (V_{H}) and variable light (V_{L}) chain region polypeptides, complementarity determining region (CDR) polypeptides, single domain antibodies, and polypeptides that contain at least a portion of an immunoglobulin that is sufficient to retain target-specific binding activity. Peptibodies, which consists of an immunoglobulin constant region doman (Fc) linked to two binding peptides through either the carboxyl- or amino termini of the Fc domain, also are included herein as an antibody functional fragment. Such antibody binding fragments can be found described in, for example, Harlow and Lane, supra; Molec. Biology and Biotechnology: A Comprehensive Desk Reference (Myers, R. A. (ed.), New York: VCH Publisher, Inc.); Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990).

With respect to antibodies and functional fragments thereof that exhibit beneficial binding characteristics to a target molecule, various forms, alterations and modifications are well known in the art. Target-specific monoclonal antibodies for use in a biopharmaceutical formulation of the invention can include any of such various monoclonal antibody forms, alterations and modifications. Examples of such various forms and terms as they are known in the art are set forth below.

A Fab fragment refers to a monovalent fragment consisting of the V_{L}, V_{H}, C_{L} and C_{H}1 domains; a F(ab′)₂ fragment is a bivalent fragment comprising two Fab fragments linked by a disulfide bridge at the hinge region but lacking the Fc; a Fd fragment consists of the V_{H} and C_{H}1 domains; an Fv fragment consists of the V_{L} and V_{H} domains of a single arm of an antibody; and a dAb fragment (Ward et al., Nature 341:544-546, (1989)) consists of a V_{H} domain.

An antibody can have one or more binding sites. If there is more than one binding site, the binding sites may be identical to one another or may be different. For example, a naturally occurring immunoglobulin has two identical binding sites, a single-chain antibody or Fab fragment has one binding site, while a “bispecific” or “bifunctional” antibody has two different binding sites.

A single-chain antibody (scFv) refers to an antibody in which a V_{L} and a V_{H} region are joined via a linker (e.g., a synthetic sequence of amino acid residues) to form a continuous polypeptide chain wherein the linker is long enough to allow the protein chain to fold back on itself and form a monovalent antigen binding site (see, e.g., Bird et al., Science 242:423-26 (1988) and Huston et al., Proc. Natl. Acad. Sci. USA 85:5879-83 (1988)). Diabodies refer to bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., Proc. Natl. Acad. Sci. USA 90:6444-48 (1993), and Poljak et al., Structure 2:1121-23 (1994)). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

A CDR refers to a region containing one of three hypervariable loops (H1, H2 or H3) within the non-framework region of the immunoglobulin (Ig or antibody) V_{H} β-sheet framework, or a region containing one of three hypervariable loops (L1, L2 or L3) within the non-framework region of the antibody V_{L} βD-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a loop varies in different antibodies, additional loop residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

TABLE
CDR Definitions

| | Kabat¹ | Chothia² | Loop Location |
|---|---|---|---|
| V_{H} CDR1 | 31-35 | 26-32 | linking B and C strands |
| V_{H} CDR2 | 50-65 | 53-55 | linking C′ and C″ strands |
| V_{H} CDR3 | 95-102 | 96-101 | linking F and G strands |
| V_{L} CDR1 | 24-34 | 26-32 | linking B and C strands |
| V_{L} CDR2 | 50-56 | 50-52 | linking C′ and C″ strands |
| V_{L} CDR3 | 89-97 | 91-96 | linking F and G strands |

¹Residue numbering follows the nomenclature of Kabat et al., supra
²Residue numbering follows the nomenclature of Chothia et al., supra

A chimeric antibody refers to an antibody that contains one or more regions from one antibody and one or more regions from one or more other antibodies. In one specific example, one or more of the CDRs are derived from a non-human donor antibody having specific activity to a target molecule and the variable region framework is derived from a human recipient antibody. In another specific example, all of the CDRs can be derived from a non-human donor antibody having specific activity to a target molecule and the variable region framework is derived from a human recipient antibody. In yet another specific example, the CDRs from more than one non-human target-specific antibodies are mixed and matched in a chimeric antibody. For instance, a chimeric antibody can include a CDR1 from the light chain of a first non-human target-specific antibody, a CDR2 and a CDR3 from the light chain of a second non-human target-specific antibody and the CDRs from the heavy chain from a third target-specific antibody. Further, the framework regions can be derived from one of the same or from one or more different human antibodies or from a humanized antibody. Chimeric antibodies can be produced where both the donor and recipient antibodies are human.

A humanized antibody or grafted antibody has a sequence that differs from a non-human species antibody sequence by one or more amino acid substitutions, deletions, and/or additions, such that the humanized antibody is less likely to induce an immune response, and/or induces a less severe immune response, as compared to the non-human species antibody, when it is administered to a human subject. In one specific example, certain amino acids in the framework and constant domains of the heavy and/or light chains of the non-human species antibody are changed to produce the humanized antibody. In another specific example, the constant domain(s) from a human antibody are fused to the variable domain(s) of a non-human species. Examples of how to make humanized antibodies may be found in U.S. Pat. Nos. 6,054,297, 5,886,152 and 5,877,293. Humanized antibodies also include antibodies produced using antibody resurfacing methods and the like.

A human antibody refers to antibodies that have one or more variable and constant regions derived from human immunoglobulin sequences. For example, a fully human antibody includes an antibody where all of the variable and constant domains are derived from human immunoglobulin sequences. Human antibodies can be prepared using a variety of methods known in the art.

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to specifically bind to a particular antigen of interest.

A neutralizing antibody or an inhibitory antibody refers to a target-specific monoclonal antibody that inhibits the binding of the target molecule to its binding partner when an excess of the target-specific monoclonal antibody reduces the amount of binding partner bound to the target. Binding inhibition can occur by at least 10%, particularly by at least about 20%. In various specific examples, the monoclonal antibody can reduce the amount of binding partner bound to the target by, for example, at least 30%, 40%, 50%, 60%, 70%, 75%, 80%, 85%, 90%, 95%, 97%, 99%, and 99.9%. The binding reduction can be measured by any means known to one of ordinary skill in the art, for example, as measured in an in vitro competitive binding assay.

An antagonistic antibody refers to an antibody that inhibits the activity of a target molecule when added to a cell, tissue or organism expressing the target molecule. Diminution in activity can be by at least about 5%, particularly by at least about 10%, more particularly, by at least about 15% or more, compared to the level of target molecule activity in the presence of binding partner alone. In various specific examples, the target-specific monoclonal antibodies for use as a biopharmaceutical of the invention can inhibit the target molecule activity by at least about 20%, 30%, 40%, 50%, 60%, 70%, 80%, 90% or 100%.

As with the above described target-specific monoclonal antibodies, in further embodiments, target-specific monoclonal antibodies for use as a biopharmaceutical of the invention include monoclonal antibodies that exhibit target molecule antagonistic activity. An antagonist of target molecule activity decreases at least one function or activity of the target molecule when bound or stimulated by its binding partner. Such functions can include, for example, stimulation or inhibition of cell regulation, gene regulation, protein regulation, signal transduction, cell proliferation, differentiation, migration, cell survival or any other biochemical and/or physiological function. Other functions or activities of a target molecule also can be reduced or inhibited by antagonistic target-specific monoclonal antibodies for use as a biopharmaceutical of the invention. Given the teachings and guidance provided herein, those skilled in the art will be able to make and identify a wide range of target-specific monoclonal antibodies exhibiting different antagonistic activities.

Antagonistic target-specific monoclonal antibodies of the invention can be produced and identified as described herein. A specific method for identifying antagonistic target-specific monoclonal antibodies includes contacting a target-specific monoclonal antibody with a target molecule expressing cell that is responsive to its binding partner in the presence of binding partner or other agonist. Contacting is performed under conditions sufficient for binding and a decrease or reduction in a target molecule function or activity can be determined. Those target-specific monoclonal antibodies that decrease, reduce or prohibit at least one function or activity of the target are identified as being a target-specific antagonistic monoclonal antibody

An agonist antibody refers to an antibody that activates a target molecule by at least about 5%, particularly by at least about 10%, more particularly, by at least about 15% when added to a cell, tissue or organism expressing the target molecule, where 100% activation is the level of activation achieved under physiological conditions by the same molar amount of binding partner. In various specific examples, the target-specific monoclonal antibodies for use as a biopharmaceutical of the invention can activate target molecule activity by at least about 20%, 30%, 40%, 50%, 60%, 70%, 80%, 90%, 100%, 125%, 150%, 175%, 200%, 250%, 300%, 350%, 400%, 450%, 500%, 750% or 1000%.

In further embodiments, target-specific monoclonal antibodies for use as a biopharmaceutical of the invention include monoclonal antibodies that exhibit target molecule agonistic activity. An agonistic of target molecule activity refers to a molecule that increases at least one function or activity of the target molecule when bound to its binding partner. Activities that can be increased include, for example, those described previously with respect to antagonistic activities. Accordingly, target-specific monoclonal antibodies having target molecule antagonist activity decrease, reduce or prevent one or more cellular functions or activities of a target molecule. Target-specific monoclonal antibodies having target molecule agonist activity increase, promote or stimulate one or more cellular functions or activities of a target molecule. Given the teachings and guidance provided herein, those skilled in the art will be able to make and identify a wide range of target-specific monoclonal antibodies exhibiting different antagonistic or agonistic activities.

Given the teachings and guidance provided herein, those skilled in the art can employ immunization methods, hybridoma production, myeloma cell-line expression and screening methods well known in the art to produce agonistic target-specific monoclonal antibodies. A specific method for identifying agonistic target-specific monoclonal antibodies includes contacting a target-specific monoclonal antibody with a target molecule expressing cell that is responsive to the target molecule binding partner under conditions sufficient for binding and determining stimulation or increase in a target molecule function or activity. Those target-specific monoclonal antibodies that increase, stimulate or promote at least one function or activity of target molecule are identified as being a target-specific agonistic monoclonal antibody.

An epitope refers to a part of a molecule, for example, a portion of a polypeptide, that specifically binds to one or more antibodies within the antigen binding site of the antibody. Epitopic determinants can include continuous or non-continuous regions of the molecule that bind to an antibody. Epitopic determinants also can include chemically active surface groupings of molecules such as amino acids or sugar side chains and have specific three dimensional structural characteristics and/or specific charge characteristics.

Specific binding refers to a target-specific monoclonal antibody exhibiting preferential binding for a target molecule compared to other related but non-target molecules or compared to other non-target molecules. Preferential binding includes a monoclonal antibody for use as a biopharmaceutical of the invention exhibiting detectable binding to its target molecule while exhibiting little or no detectable binding to another related but non-target molecule.

Specific binding can be determined by any of a variety of measurements known to those skilled in the art including, for example, affinity (Kₐ or K_{d}), association rate (kₒₙ), dissociation rate (k_{off}), avidity or a combination thereof. Any of a variety of methods or measurements well known in the art can be employed and are applicable for determining target-specific binding activity. Such methods and measurements include, for example, apparent or relative binding between a target molecule and a non-target molecule. Both quantitative and qualitative measurements can be employed for making such apparent or relative binding determinations. Specific examples of binding determinations include, for example, competitive binding assays, protein or Western blot methodology, ELISA, RIA, surface plasmon resonance, evanescent wave methodology, flow cytometry and/or confocal microscopy.

Further, specific binding of antagonistic or agonistic target-specific monoclonal antibodies can be determined by any of the methods described above or below including, for. example, determining a change in a cellular function or activity. Methods for measuring a change in cellular function or activity such as proliferation, differentiation or other biochemical and/or physiological function are well known in the art. As with the binding assays described previously, both quantitative and qualitative measurements can be employed for making apparent or relative determinations with respect to antagonizing or agonizing one or more cellular functions.

Target-specific monoclonal antibodies for use as a biopharmaceutical of the invention, or functional fragments thereof, can be produced in any of the various antibody forms and/or can be altered or modified in any of the various ways as described previously while still maintaining their specific target binding activity. Any of such antibody forms, alterations or modifications, including combinations thereof, of a target-specific monoclonal antibody, or functional fragment thereof, is included within the invention as a biopharmaceutical. Any of such various antibody forms, alterations or modifications of a target-specific monoclonal antibody for use as a biopharmaceutical of the invention, or a functional fragment thereof, can similarly be used in the methods, compositions and/or articles of manufacture of the invention as they are described herein. For example, target-specific monoclonal antibodies of the invention, or functional fragments thereof, include target-specific grafted, humanized, Fd, Fv, Fab, F(ab)₂, scFv and peptibody monoclonal antibodies as well as all other forms, alterations and/or modifications described previously, and including other forms well known to those skilled in the art.

Methods for producing hybridomas and screening for target-specific monoclonal antibodies using hybridoma technology are routine and well known in the art. For example, mice can be immunized with a target molecule such as a polypeptide and once an immune response is detected, e.g., antibodies specific for the target molecule are detected in the mouse serum, the mouse spleen is harvested and splenocytes isolated. The splenocytes are then fused by well known methods to any suitable myeloma cells, for example, cells from cell line SP20 available from the ATCC. Hybridomas are selected and cloned by limited dilution. The hybridoma clones are then assayed by methods known in the art for cells that secrete antibodies capable of binding a target molecule. Ascites fluid, which generally contains high levels of antibodies, can be generated by immunizing mice with positive hybridoma clones.

Additionally, recombinant expression in prokaryotic or eukaryotic hosts can be used to generate target-specific monoclonal antibodies. Recombinant expression can be utilized to produce single target-specific monoclonal antibody species, or functional fragments thereof. Alternatively, recombinant expression can be utilized to produce diverse libraries of heavy and light, or variable heavy and variable light chain combinations, and then screened for a monoclonal antibody, or functional fragment thereof, exhibiting specific binding activity to the target molecule. For example, heavy and light chains, variable heavy and light chain domains, or functional fragments thereof, can be co-expressed from nucleic acids encoding target-specific monoclonal antibodies using methods well known in the art to produce specific monoclonal antibody species. Libraries can be produced using methods well known in art from co-expressed populations of nucleic acids encoding heavy and light chains, variable heavy and light chain domains, or functional fragments thereof, and screened by affinity binding to the target molecule for identification of target-specific monoclonal antibodies. Such methods can be found described in, for example, Antibody Engineering: A Practical Guide, C. A. K. Borrebaeck, Ed., supra; Huse et al., Science 246:1275-81 (1989); Barbas et al., Proc. Natl. Acad. Sci. USA 88:7978-82 (1991); Kang et al., Proc. Natl. Acad. Sci. USA 88:4363-66 (1991); Plückthun and Skerra, supra; Felici et al., J. Mol. Biol. 222:301-310 (1991); Lerner et al., Science 258:1313-14 (1992), and in U.S. Pat. No. 5,427,908.

Cloning of encoding nucleic acids can be accomplished using methods well known to those skilled in the art. Similarly, cloning of heavy and/or light chain repertoires of encoding nucleic acid, including V_{H} and/or V_{L} encoding nucleic acids also can be accomplished by methods well known to those skilled in the art. Such methods include, for example, expression cloning, hybridization screening with a complementary probe, polymerase chain reaction (PCR) using a complementary pair of primers or ligase chain reaction (LCR) using a complementary primer, reverse transcriptase PCR (RT-PCR) and the like. Such methods can be found described in, for example, Sambrook et al., Molecular Cloning: A Laboratory Manual, Third Ed., Cold Spring Harbor Laboratory, New York (2001) and Ansubel et al., Current Protocols in Molecular Biology, John Wiley and Sons, Baltimore, Md. (1999).

Encoding nucleic acids also can be obtained from any of various public databases including whole genome databases such as those operated by The National Center for Biotechnology Information (NCBI) of the National Institutes of Health (NIH). A particularly useful method of isolating either a single encoding nucleic or a repertoire of encoding nucleic acids for heavy and/or light chains, or functional fragments thereof, can be accomplished without specific knowledge of the coding region portion because primers are available or can be readily designed using conserved portions of antibody variable or constant region portions. For example, a repertoire of encoding nucleic acids can be cloned using a plurality of degenerate primers to such regions together with PCR. Such methods are well known in the art and can be found described in, for example, Huse et al., supra, and Antibody Engineering: A Practical Guide, C. A. K. Borrebaeck, Ed., supra. Any of the above methods as well as others known in the art, including combinations thereof, can be used to generate a target-specific monoclonal antibody for use as a biopharmaceutical of the invention.

Therefore, the invention provides a biopharmaceutical formulation having an antibody, a functional fragment of an antibody as a therapeutic polypeptide. The therapeutic polypeptide can include a monoclonal antibody, Fd, Fv, Fab, F(ab′), F(ab)₂, F(ab′)₂, single chain Fv (scFv), chimeric antibodies, diabodies, triabodies, tetrabodies, minibody or peptibody.

Concentrations of a biopharmaceutical to be included in a formulation of the invention will vary, for example, depending on the activity of the biopharmaceutical, the indication to be treated, mode of administration, the treatment regime and whether the formulation is intended for long term storage in either liquid or lyophilized form. Those skilled in the art will know what concentrations to use given these well known considerations and the state of the art in the pharmaceutical sciences. For example, there are more than 80 biopharmaceuticals approved for therapeutic use in the United States for a wide range of medical indications, modes of administration and treatment regimes. These approved biopharmaceuticals are exemplary of the range of biopharmaceutical concentrations that can be used in a biopharmaceutical formulation of the invention.

Generally, a biopharmaceutical including, for example, a therapeutic polypeptide biopharmaceutical, will be included in a formulation of the invention at a concentration from between about 1-200 mg/ml, about 10-200 mg/ml, about 20-180 mg/ml, particularly between about 30-160 mg/ml, more particularly between about 40-120 mg/ml, even more particularly between about 50-100 mg/ml or about 60-80 mg/ml. Biopharmaceutical concentrations and/or amounts less than, greater than or in between these ranges also can be used in a biopharmaceutical formulation of the invention. For example, one or more biopharmaceuticals can be included in a biopharmaceutical formulation which constitute less than about 1.0 mg/ml. Similarly, a biopharmaceutical formulation can contain a concentration of one or more biopharmaceuticals greater than about 200 mg/ml, particularly when formulated for storage. Accordingly, a biopharmaceutical formulation of the invention can be produced that contains essentially any desired concentration or amount of one or more biopharmaceuticals including, for example, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190 or 200 mg./ml or more. Exemplified in the Example below is a biopharmaceutical formulation for a therapeutic polypeptide having a concentration of about 10 mg/ml.

A biopharmaceutical formulation of the invention also can include combinations of biopharmaceuticals in the formulation. For example, a biopharmaceutical formulation of the invention can include a single biopharmaceutical for treatment of one or more conditions. A biopharmaceutical formulation of the invention also can include two or more different biopharmaceuticals. Use of multiple biopharmaceuticals in a formulation of the invention can be directed to, for example, the same or different indications. Similarly, multiple biopharmaceuticals can be used in a formulation of the invention to treat, for example, both a pathological condition and one or more side effects caused by the primary treatment. Multiple biopharmaceuticals also can be included in a formulation of the invention to accomplish different medical purposes including, for example, simultaneous treatment and monitoring of the progression of the pathological condition. Multiple, concurrent therapies such as those exemplified above as well as other combinations well known in the art are particularly useful for patient compliance because a single formulation can be sufficient for some or all suggested treatments and/or diagnosis. Those skilled in the art will know those biopharmaceuticals that can be admixed for a wide range of combination therapies. Similarly, a biopharmaceutical formulation of the invention also can be used with small molecule pharmaceuticals and combinations of one or more biopharmaceuticals together with one or more small molecule pharmaceuticals. Therefore, the invention provides for a biopharmaceutical formulation of the invention containing 1, 2, 3, 4, 5 or 6 or more different biopharmaceuticals as well as for one or more biopharmaceuticals combined with one or more small molecule pharmaceuticals.

A biopharmaceutical formulation of the invention also can include one or more preservatives and/or additives well known in the art. Similarly, a biopharmaceutical formulation of the invention can further be formulated into any of various know delivery formulations. For example, a biopharmaceutical formulation of the invention can include lubricating agents, emulsifying agents, suspending agents, preserving agents such as methyl- and propylhydroxy-benzoates, sweetening agents and flavoring agents. Such optional components, their chemical and functional characteristics are well known in the art. Similarly well known in the art are formulations that facilitate rapid, sustained or delayed release of the biopharmaceutical after administration. A biopharmaceutical formulation of the invention can be produced to include these or other formulation components well known in the art.

A biopharmaceutical formulation of the invention also can be produced, for example, in states other than an aqueous liquid. As described previously, propionic acid is less volatile relative to certain other week acids. For example, propionate has a vapor pressure (VP) of 3.3 mm Hg at 28° C. is less volatile compared to acetate which has a VP of 11 mm Hg at 20° C. It is contemplated that this lower volatility can be particularly useful in preparing a lyophilized formulation because more of the formulation components can be retained during the lyophilization process, resulting in less risk for desorption.

Once a biopharmaceutical formulation of the invention is prepared as described herein, stability of the one or more biopharmaceuticals contained within the formulation can be assessed using methods well known in the art. Several of such methods are exemplified further below in the Examples and include size exclusion chromatography, particle counting and osmolality. Any of a variety of functional assays including, for example, binding activity, other biochemical activity and/or physiological activity can be assessed at two or more different time points to determine the stability of the biopharmaceutical in the buffered formulation of the invention.

A biopharmaceutical formulation of the invention will, in general, be prepared according to pharmaceutical standards and using pharmaceutical grade reagents. Similarly, a biopharmaceutical formulation of the invention will, in general, be prepared using sterile reagents in a sterile manufacturing environment or sterilized following preparation. Sterile injectable solutions can be prepared using well known procedures in the art including, for example, by incorporating one or more biopharmaceuticals in the required amount in a propionic acid buffer or excipient of the invention with one or a combination of formulation components described herein followed by sterilization microfiltration. In the specific embodiment of sterile powders for the preparation of sterile injectable solutions, particularly useful methods of preparation include, for example, vacuum drying and freeze-drying (lyophilization) as described previously. Such drying methods will yield a powder of the one or more biopharmaceuticals together with any additional desired components from a previously sterile-filtered solution thereof.

Administration and dosage regimens can be adjusted to provide an effective amount for an optimum therapeutic response. For example, a single bolus can be administered, several divided doses can be administered over time or the dose can be proportionally reduced or increased as indicated by the exigencies of the therapeutic situation. It can be particularly useful to formulate a biopharmaceutical formulation of the invention for intravenous, parenteral or subcutaneous injection in a unit dosage form for ease of administration and uniformity of dosage in administering an effective amount of one or more biopharmaceuticals. Unit dosing refers to a physically discrete amount of pharmaceutical suited as unitary dosages for the subjects to be treated; each unit contains a predetermined quantity of active biopharmaceutical calculated to produce a desired therapeutic effect.

For further exemplification, an effective amount of a polypeptide biopharmaceutical such as a therapeutic antibody, or functional fragment thereof, can be administered, for example, more than once, at scheduled intervals over a period of time. In certain embodiments, a therapeutic antibody is administered over a period of at least a month or more including, for example, one, two, or three months or longer. For treating chronic conditions, long-term, sustained treatment is generally most effective. Shorter periods of administration can be sufficient when treating acute conditions including, for example, from one to six weeks. In general, a therapeutic antibody or other biopharmaceutical is administered until the patient manifests a medically relevant degree of improvement over baseline for the chosen indicator or indicators.

Depending on the selected biopharmaceutical and indication to be treated, a therapeutically effective amount is sufficient to cause a reduction in at least one symptom of the targeted pathological condition by at least about 5%, 10%, 15%, 20%, 25%, 30%, 35%, 40%, 45%, 50%, 55% or 60% or more, relative to untreated subjects. The ability of a biopharmaceutical formulation to reduce or inhibit a symptom can be evaluated, for example, in an animal model system predictive of efficacy for the targeted condition in human. Alternatively, the ability of a biopharmaceutical formulation to reduce or inhibit a symptom can be evaluated, for example, by examining an in vitro function or activity of the biopharmaceutical formulation indicative of in vivo therapeutic activity.

Actual dosage levels of one or more biopharmaceuticals in a biopharmaceutical formulation of the invention can be varied so as to obtain an amount of the active biopharmaceutical which is effective to achieve the desired therapeutic response for a particular patient, formulation, and mode of administration, without being toxic to the patient. One skilled in the art would be able to determine administered amounts based on factors such as the subject's size, the severity of the subject's symptoms, and the selected biopharmaceutical and/or route of administration. The selected dosage level can depend, for example, upon a variety of pharmacokinetic factors including the activity of the biopharmaceutical employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts. Particular embodiments of the present invention involve administering a therapeutic polypeptide such as an antibody, or functional fragment thereof, in a biopharmaceutical formulation of the invention at a dosage of from about 1 ng of antibody per kg of subject's weight per day (1 ng/kg/day) to about 10 mg/kg/day, more particularly from about 500 ng/kg/day to about 5 mg/kg/day, and even more particularly from about 5 μg/kg/day to about 2 mg/kg/day, to a subject.

A physician or veterinarian having skill in the art can readily determine and prescribe the effective amount of the required pharmaceutical formulation. For example, the physician or veterinarian can initiate doses of a biopharmaceutical formulation of the invention at levels lower than that required in order to achieve the desired therapeutic effect and gradually increase the dosage until the desired effect is achieved. In general, a suitable daily dose of a biopharmaceutical formulation of the invention will be that amount of the biopharmaceutical which is the lowest dose effective to produce a therapeutic effect. Such an effective amount will generally depend upon the factors described previously. It is particularly useful that administration be intravenous, intramuscular, intraperitoneal, or subcutaneous. If desired, the effective daily dose to achieve an effective amount of a biopharmaceutical formulation can be administered as two, three, four, five, six or more sub-doses administered separately at appropriate intervals throughout the day, optionally, in unit dosing amounts.

A biopharmaceutical formulation of the invention can be administered, for example, with medical devices known in the art. For example, in a particularly useful embodiment, a biopharmaceutical formulation of the invention can be administered with a needleless hypodermic injection device, such as the devices described in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which describes an implantable micro-infusion pump for dispensing medication at a controlled rate; U.S. Pat. No. 4,486,194, which describes a therapeutic device for administering medicants through the skin; U.S. Pat. No. 4,447,233, which describes a medication infusion pump for delivering medication at a precise infusion rate; U.S. Pat. No. 4,447,224, which describes a variable flow implantable infusion apparatus for continuous drug delivery; U.S. Pat. No. 4,439,196, which describes an osmotic drug delivery system having multi-chamber compartments, and U.S. Pat. No. 4,475,196, which describes an osmotic drug delivery system. Many other such implants, delivery systems, and modules are known to those skilled in the art.

In certain specific embodiments, a biopharmaceutical for use in a formulation of the invention can additionally be formulated to facilitate selective distribution in vivo. For example, the blood-brain barrier (BBB) excludes many highly hydrophilic compounds. To facilitate crossing of the BBB if desired, a biopharmaceutical formulation can additionally include, for example, liposomes for encapsulation of one or more biopharmaceuticals. For methods of manufacturing liposomes, see, for example, U.S. Pat. Nos. 4,522,811; 5,374,548; and 5,399,331. The liposomes can further contain one or more moieties which are selectively transported into specific cells or organs, thus enhancing targeted delivery of a selected biopharmaceutical (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180) or surfactant protein A receptor (Briscoe et al. (1995) Am. J. Physiol. 1233:134).

Therefore, the invention additionally provides a method of preparing a biopharmaceutical formulation. The method includes combining an aqueous solution having a propionate buffer having a pH from about 4.0 to about 6.0, an excipient and a surfactant with an effective amount of a therapeutic polypeptide. One or more of the biopharmaceutical formulation components described herein can be combined with one or more effective amounts of a biopharmaceutical to produce a wide range of formulations of the invention.

Additionally provided is a container containing a biopharmaceutical formulation including an aqueous solution having between about 3-20 mM propionate with a pH from about 4.0 to about 6.0, sorbitol between about 1-10%, polysorbate 20 between about 0.001-0.010% and an effective amount of a therapeutic polypeptide. Briefly, with respect to compositions, kits and/or medicaments of the invention, the combined effective amounts of one or more biopharmaceuticals within a formulation of the invention can be included within a single container or container means, or included within distinct containers or container means. Imaging components can optionally be included and the packaging also can include written or web-accessible instructions for using the biopharmaceutical formulation. A container or container means includes, for example, a vial, bottle, syringe or any of a variety of formats well known in the art for multi-dispenser packaging.

It is understood that modifications which do not substantially affect the activity of the various embodiments of this invention are also included within the definition of the invention provided herein. Accordingly, the following examples are intended to illustrate but not limit the present invention.

### EXAMPLE I

### Polypeptide Stability Characterization in Buffered Aqueous Solutions

This Example describes the characterization of various formulation components and formulations on the stability of therapeutic polypeptides.

Epratuzumab (Emab) is a humanized recombinant monoclonal antibody (mAB) expressed in myeloma cells. It has a pI in the range of 9.12 to 9.27 and has been shown to have therapeutic efficacy against non-Hodgkins lymphoma (NHL). Emab binds CD22, a B-cell surface antigen, which is expressed by a majority of B-cell NHL's. CD22 appears to be involved in the regulation of B-cell activation through the B-cell receptor.

Emab exhibits a tendency to form insoluble particulates when formulated in phosphate buffered saline (PBS; 40 mM sodium phosphate (pH 7.4), 140 mM NaCl; Immunomedics, Inc., Morris Plains, N.J.). Currently average doses of 72 mL (at 10 mg/mL) are administered to patients via IV infusion and can extend over a period of about 1 hour. Because of the tendency to form particulates, it is necessary to use an inline filter during IV infusion. This Example describes the generation and characterization of a biopharmaceutical formulation that augments retention in the stability of Emab and reduces the amount of particle formation. The biopharmaceutical formulation characterization with respect to Emab is exemplary of other polypeptides and biopharmaceuticals.

The formulation studies described herein were directed to three primary areas: (1) preformulation characterization, (2) formulation optimization, and (3) selection of formulations that augment retention in stability. Briefly, preformulation refers to the characterization of optimal pH, buffer, excipient and salt conditions that confer a stable product. Optimizing the pH involved profiling melting temperature behavior (Tm) using differential scanning calorimetry (DSC) in addition to carrying out accelerated studies in vials over a short period of time to confirm and decipher instability as a function of pH condition. Formulation optimization involved optimizing key excipient or stabilizer composition within the formulation and selecting those candidates with beneficial characteristics to evaluate in real-time stability studies in appropriate container and closure presentations used in the clinic or for commercial production. Real-time refers to evaluation at the recommended temperature condition that the biopharmaceutical product will be stored (generally refrigerated conditions). Finally, the selection process refers to choosing 3 or more candidates in the appropriate dosage form that offer the greatest combination of characteristics for promoting stability for up to two years under real-time conditions.

Based on preliminary findings of the preformulation work, three candidate formulations were selected for long-term stability evaluations at −80 C, 2-8 C (real-time), and 37 C (accelerated) conditions. The characterization of these candidate formulations are exemplified below by reference to the 6-month timepoint results. Results for long-term, real-time and accelerated conditions also are exemplified below.

Preformulation Results

Optimal pH profiles for stability of the exemplary Emab polypeptide were assessed using differential scanning calorimetry (DSC) thermal heating studies. Briefly, these DSC studies were performed on Emab in polypeptide buffers having a range of pH from 3.5 to 11. High relative melting temperatures (Tm) indicate regions of conformational stability within a polypeptide (Remmele, R. L., Jr., (2005) “Microcalorimetic Approaches to Biopharmaceutical Development”, in Analytical Techniques for Biopharmaceutical Development (Rodriguez-Diaz, R., Wehr, T., Tuck, S., eds.), Marcel/Dekker, New York, N.Y., pp. 327-381 (ISBN:0-8247-0706-0)).

The DSC results indicated that the optimal pH range as it related to buffer conditions where the most predominant DSC peak resulted in the highest Tm's occurred between pH 6 and 9. The highest Tm condition was obtained at pH 6. These results indicate that the polypeptide conformation was most stable and less prone to unfold within this pH range of 6-9.

Accelerated stability studies were performed to characterize the form and type of degradation observed within the studied range of pH conditions. Briefly, accelerated stability studies performed at a particular pH and at, for example, 37 C using size exclusion chromatography (SEC). A TosoHaas G3000SWxl dual column in tandem was used to carry out the analysis using a mobile phase consisting of 50 mM phosphate (pH 7), 250 mM NaCl and 5% ethanol. Samples were dialyzed into the respective formulations to be tested and sterile filtered into sterile containers. Approximately 2-mL quantities of each formulated sample were placed in sterile 3-mL glass vials in a sterile hood and stoppered. Samples designated for freezing were placed in sterile polypropylene cryo-tubes. All vials were labeled and capped followed by placement into boxes specified for storage at −80 C, 2-8 C, and 37 C conditions. Samples were removed and analyzed at designated timepoints.

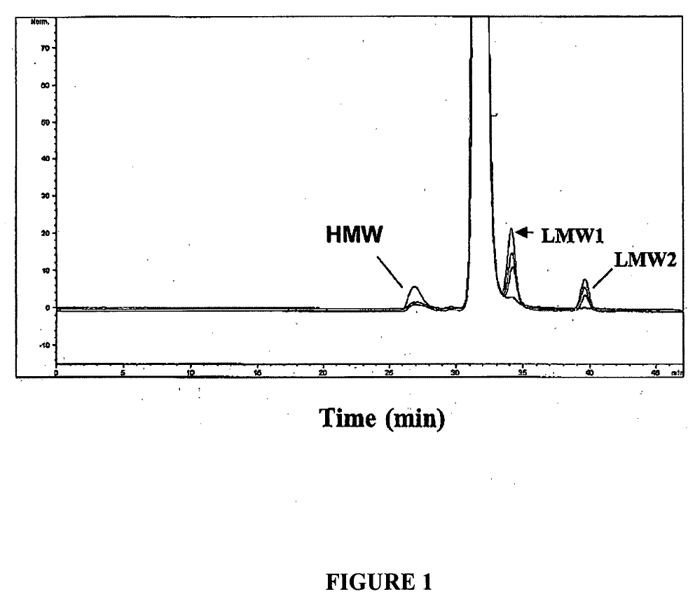

Different forms of the samples could be quantitatively evaluated and separated based on their hydrodynamic volume. Exemplary results are illustrated in FIG. 1 and show soluble aggregates (designated by HMW) and low molecular weight fragments (designated by LMWI and LMW2) that increase during heating for one month at 37 C. The HMW peak is a dimer of the main peak or intact antibody and has been characterized (Remmele, et al., (2006) Active dimer of Epratuzumab provides insight into the complex nature of an antibody aggregate.; J Pharm Sci. January 2006;95(1):126-145).

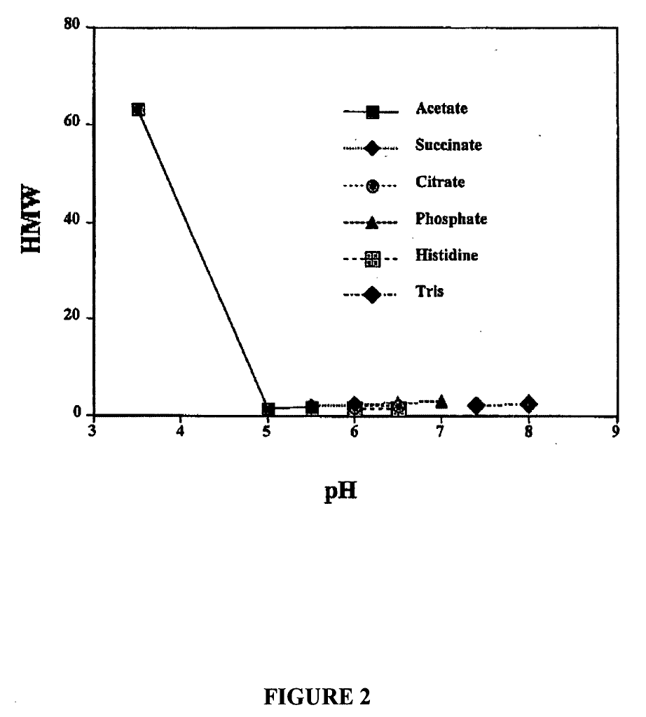

The influence of pH and buffer on the high molecular weight (HMW) and low molecular weight (LMW) instability pathways were assessed by plotting the formation of HMW and LMW fragments as a function of pH. The HMW results are shown in FIG. 2 and indicate a sharp decline in soluble aggregate formation moving from pH 3.5 to 5. Increasing the pH beyond pH 5 showed that aggregate formation was low and relatively flat within the range of the Tm optimum determined by DSC and described above. FIG. 2 also shows that there was little distinction between the different buffers and acquired benefit as it pertained to the formation of HMW species.

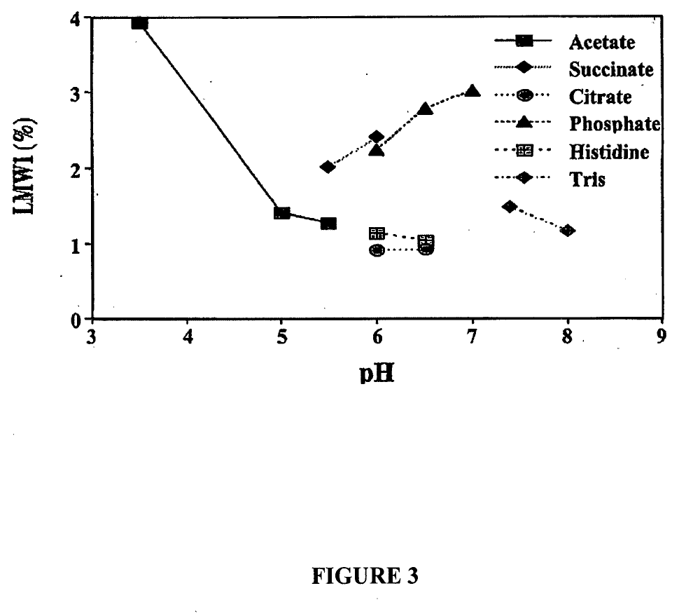

FIG. 3 shows the behavior of LMW1 with respect to pH conditions and the affects of different buffering agents on the propensity of the polypeptide to fragment. As shown, a greater amount of fragmentation was observed between pH 3.5 and 5 than at higher pH conditions. However, some of the buffer agents screened appeared to increase the fragmentation process particularly, for example, succinate and phosphate. The rank order from most to least effective regarding influence on the stabilization against breakdown is illustrated below.
Citrate>Histidine>[Tris˜Acetate]>[Phosphate˜Succinate]

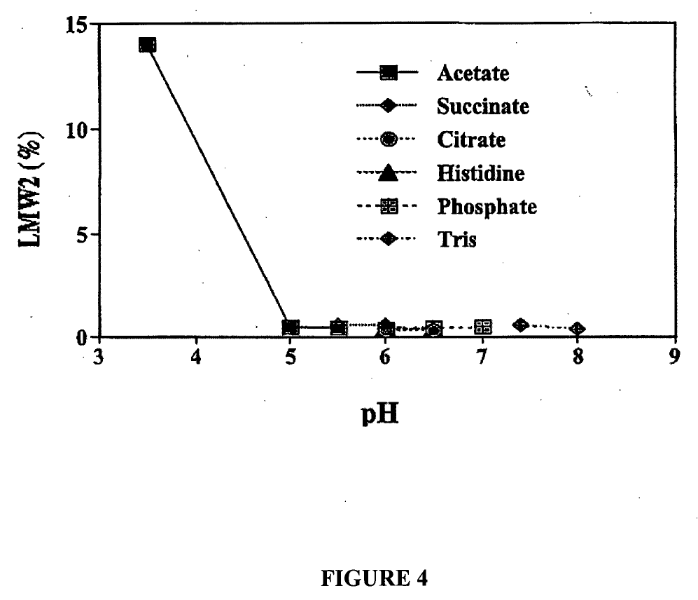

The same evaluation was performed for the LMW2 fragment. The results are shown in FIG. 4 and indicate a similar pH behavior as observed for the HMW and LMW1 forms. For example, minimal fragmentation of the polypeptide occurs within the range from pH 5 to 8. Similarly, there was little difference between the buffer systems studied. In other studies, inclusion of divalent metals such as copper was found to induce the formation of LMW species.

As described further below, different excipients also were preliminarily screened for optimal polypeptide stability characteristics. Briefly, Emab samples were formulated in PBS, concentrated and dialyzed into 23 primary buffer solutions and then diluted to a desired volume. EDTA was added to reach differing final concentrations not exceeding 5 mM in selected formulations. Samples were subsequently sterile filtered, and a 2-mL volume placed into a sterile 5 cc glass vial in a sterile hood. All vials were stoppered, labeled and caped prior to placement in boxes designated for storage at 4 C and 37 C. Samples were removed and analyzed at designated time points.

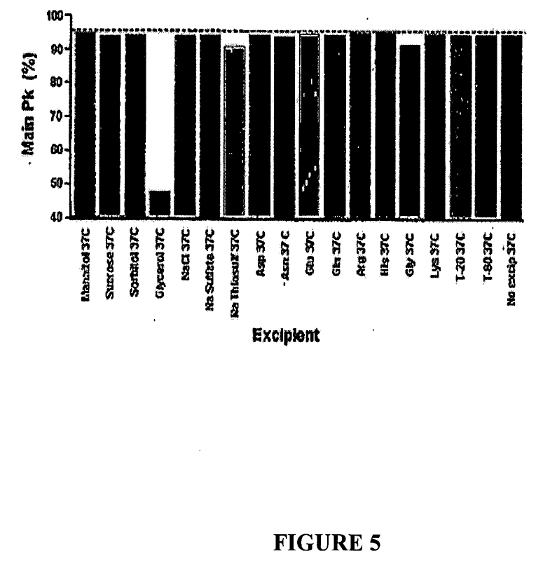

FIG. 5 shows the effect of 18 different excipients evaluated at 37 C. Glycerol was found to be the most destabilizing, generating ˜51% total HMW species. Glycine and sodium thiosulfate were the next most destabilizing. The Glycine formulations resulted in about 3% total HMW species with about a 5% breakdown contributing to the loss in main peak. Thiosulfate on the other hand, exhibited about 8% breakdown predominantly accounting for the loss in main peak for that sample. The most stabilizing excipients included mannitol, L-arginine, L-lysine, sorbitol, sucrose, Tween-80 and Tween-20. The remaining excipients showed little change when compared with each other. Excipient concentrations used in the study are listed below in Table 1.

TABLE 1
Excipient Concentrations used in
Preliminary Optimization Studies

| Excipient | Concentration | |
|---|---|---|
| Mannitol | 5% | (w/v) |
| Sucrose | 5% | (w/v) |
| Sorbitol | 5% | (w/v) |
| Glycerol | 5% | (w/v) |
| NaCl | 150 | mM |
| Sodium Sulfate | 25 | mM |
| Sodium Thiosulfate | 25 | mM |
| L-Aspartic Acid | 12 | mM |
| L-Asparagine | 12 | mM |
| L-Glutamic Acid | 12 | mM |
| L-Glutamine | 12 | mM |
| L-Arginine | 25 | mM |
| L-Histidine | 25 | mM |
| L-Glycine | 25 | mM |
| L-Lysine | 25 | mM |
| Tween-20 | 0.05% | |
| Tween-80 | 0.05% | |
| Control (No Excipients) | | |

One characteristic relating to Emab and other polypeptides is the occurrence of subvisible insoluble particles. In this context, a polypeptide particle refers to, for example, a fragment or aggregate of the polypeptide and can be soluble and/or insoluble. Additionally, particles can be made up of matter that is foreign (i.e., shards of glass, lint, small pieces of rubber stopper) and not necessarily composed of the polypeptide. Soluble particles can be evaluated using methods such as SEC, for example. Particles that are insoluble can be evaluated using such methods as liquid particle counting or turbidimetric techniques (empirical light scattering approach), for example. Coarse particles are generally classified as particles having sizes greater than 1.0 μm and those considered fine particles are smaller in size. Using the LD-400 laser system with the HIAC instrument (Geneva, Switzerland), particle sizes between 2 and 400 μm can be measured.

As shown previously with reference to FIG. 1, in addition to formation of insoluble particulates aqueous solutions of Emab also breakdown or fragment into two predominant species designated LMW1 and LMW2. The breakdown products can be characterized with a correlation plot indicating the interdependence of LMW1 with LMW2 (percent change of LMW1 vs. LMW2). Such a plot was constructed where the change of each designated peak representing the difference in pre- and post-incubated (37 C for 2 weeks) peak area derived from the above SEC studies in a variety of different solution environments were represented. A scatter plot also was constructed showing the residual distribution of data points from that predicted by the line defined by the linear least squares fit of the data in the correlation plot. The correlation plot showed LMW1=2.17399 (LMW2)+06776, p<0.0001, and r²=0.946. A mean response of 0.5155 was obtained. Emab cleavage sites resulting in the observed degradation fragments were characterized and revealed a series of six clip sites within the polypeptide sequence, S₂₁₈CDKTHTC₂₂₅.

Preliminary stability studies also were performed to characterize optimal buffer components and conditions for polypeptide formulation. Briefly, initial formulations were selected based upon the above described preformulation studies which combined components and buffer conditions identified to impart optimal stability on polypeptide stability. These initial formulations were compared with a 10 mM sodium acetate (pH 5), 5% sorbitol formulation (A5S formulation) previously found to afford some stabilizing influence and is the current formulation of Emab. A candidate optimal formulation also was selected and consisted of 20 mM sodium phosphate (pH 6), 25 mM L-arginine, 1% sucrose, 4% mannitol, and 0.02% Tween-20 (PASMT formulation). This candidate optimal formulation provided buffer capacity in the range of the optimal pH described by the DSC profile; included Tween-20, a surfactant to stabilize against water/air interface induced aggregation which could ultimately lead to particle formation; and had mannitol and sucrose stabilizers in correct proportion to permit lyophilization. All formulated samples were placed on real-time and accelerated stability to evaluate the impact of thermal stress on product stability as described further below.

Formation of insoluble particles was assessed for each of the initial formulations using liquid particle counting. The HIAC particle counter instrument was equipped with PharmSpec software version 1.4, required to measure the 10 μm and 25 μm particles present in a given Emab sample. The employed methods followed procedures complying with USP requirements of particle assessment and quality. Filtered water (0.22 micron) was drawn through a stainless steal tube using 1.0 mL volumes and flushed approximately 10 times between sample measurements. Duke scientific EZY-CAL liquid particle 10 μm size standard was used to verify proper calibration of the instrument. Both sample and standard measurements were taken with a volume of 0.2 mL, drawn 4 times, discarding the first run and averaging the last two or three. The samples were drawn from their original vials, with a slight swirl given to each sample prior to measurement to ensure uniform mixing of the solution. The standard was vigorously shaken prior to measurement.

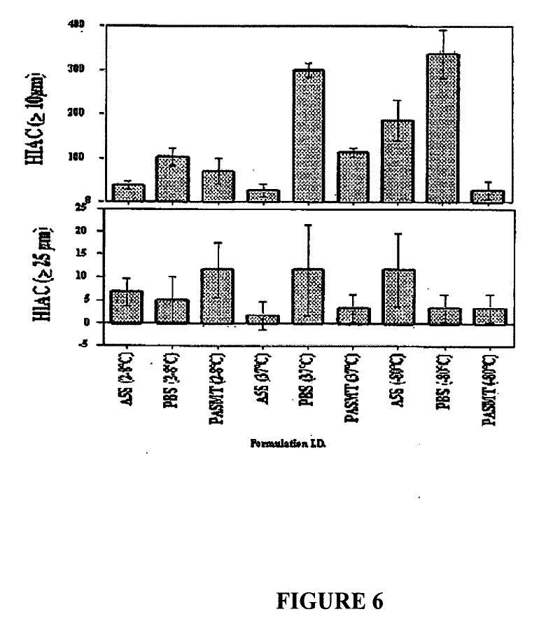

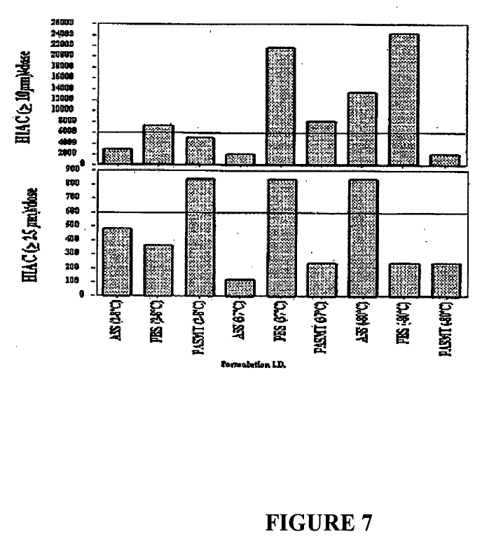

The results of the HIAC particle counts are shown in FIGS. 6 and 7. The data presented in FIG. 6 illustrate the measured assessment on a per mL basis, while the data of FIG. 7 provide an assessment of particles on a per dose basis. In the latter case, an average dose of 72 mL was used in the calculation. The data of FIG. 7 illustrate that samples formulated in the PBS formulation at all temperature conditions tested, failed to meet the >10 μm USP requirement (<6000 particles), while only the accelerated (37 C) PBS sample failed the >25 μm requirement (<600 particles). The accelerated sample formulated in PASMT failed the >10 μm requirement in contrast to the 2-8 C refrigerated sample in the same formulation that did not pass the >25 μm specification. The A5S formulation failed to pass the USP specifications for both particle counts when frozen at −80 C. This latter result indicates that Emab is subject to instability resulting in insoluble particles when stored over time at −80 C. Hence, although A5S is a good formulation for 2-8 C and accelerated conditions, it is a poor formulation for long-term storage at −80 C.

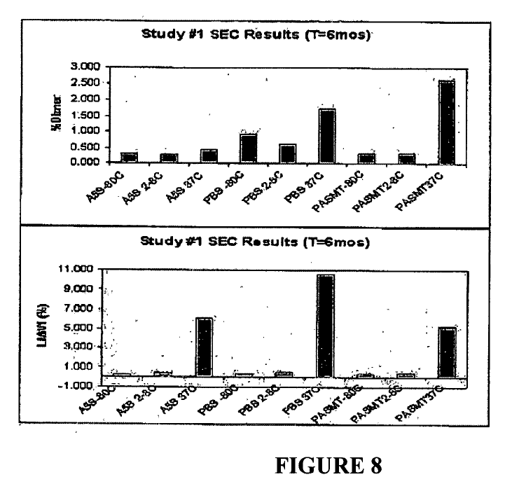

SEC also was performed with the initial formulations to assess soluble particle formation. The results are presented in FIG. 8 and reveal incongruent results in some formulations tested. For example, the dimer content of the A5S formulation was small at −80 C but the HIAC data indicated a tendency to form insoluble particulates (see FIG. 7). The PASMT formulation exhibited the most soluble dimer at 37 C, but showed better results than the PBS formulation at the same conditions with regard to insoluble particles. The PASMT formulation resulted in the best overall stability at −80 C in terms of both soluble and insoluble particles whereas the A5S formulation performed best at accelerated and real-time conditions in terms of soluble and insoluble particles. These results indicate that different mechanisms might be at play regarding particles and their relationship to dimer content. In summary, the A5S formulation offered superior protection against particles when stored at accelerated or real-time conditions, but not so favorable for freezing Emab.

As described previously with reference to FIG. 1, polypeptide fragmentation can be evaluated with reference to the predominant LMW1 peak area changes. Because LMW1 fragmentation is directly related to the LMW2 peak, as described previously with reference to the correlation and scatter plots, fragmentation tendencies also can be characterized based upon this peak alone. However, the results of all the SEC peaks analyzed are listed below in Table 2.

TABLE 2
Size-Exclusion Results for the 6-Month
Timepoint of Stability Studies

| Formulation | % HMW | % Dimer | % Main Pk | % LMW1 |
|---|---|---|---|---|
| PBS (−80 C.) | 0.918 | 0.918 | 98.748 | 0.334 |
| PBS (2-8 C.) | 0.615 | 0.615 | 9.821 | 0.488 |
| PBS (37 C.) | 2.316 | 1.736 | 82.537 | 10.554 |
| A5S (−80 C.) | 0.310 | 0.310 | 99.394 | 0.296 |
| A5S (2.8 C.) | 0.273 | 0.918 | 99.243 | 0.430 |
| A5S (37 C.) | 0.738 | 0.615 | 90.455 | 6.061 |
| PASMT (−80 C.) | 0.322 | 1.736 | 99.360 | 0.318 |
| PASMT (2-8 C.) | 0.319 | 0.310 | 99.216 | 0.416 |
| PASMT (37 C.) | 3.755 | 0.273 | 88.758 | 5.185 |

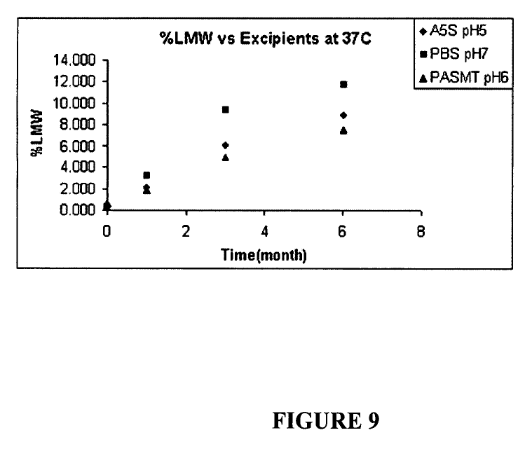

The above results indicate that among the accelerated samples, the PBS formulation exhibited the most breakdown in a 6-month period followed by the A5S and finally the PASMT formulation. The fragmentation reaction can be related to pH condition. For example, it appeared that both pH and excipients effected the fragmentation rate. In general, between pH 5, 6, and 7 conditions in the formulations tested, pH 6 was more favorable with regard to minimizing fragmentation (described further below) suggesting that conditions near pH 6 can be optimal for minimizing breakdown. This fragmentation behavior is summarized in FIG. 9 for the three formulations tested at 37 C for a 6 months incubation period. With respect to the role of the excipients in the tested formulations and their impact on the fragmentation reaction, the PASMT formulation at pH 6 yielded less fragmentation than the A5S formulation. Additional effects of excipients on fragmentation is described further below.

The cleavage sites for the fragments in the PBS formulation at 37 C at two months were isolated and characterized by LC/MS. Initial results indicated that the fragments originated from the hinge region of the antibody, and that a series of cleavage sites occurred within the peptide sequence S₂₁₈CDKTHTC₂₂₅ (see also correlation and scatter plot description above).

The bioactivity of the LMW1, LMW2 and HMW particles also was characterized. Briefly, a cell-based bioassay for determining the potency of Emab was used for examining the activity of the dimer and monomer samples. Exposure of the Ramos B-lymphoma cell-line to Emab can lead to apoptosis in 24 hrs, followed by a decrease in viable cells in 72 hrs. The decrease in viable cell content over time can be measured using an Alamar Blue fluorescent reagent. Different concentrations of Emab were immobilized on 96-well immuno plates with subsequent addition of a fixed amount of anti-IgM and Ramos cells at 2500 cells per well. The sample plate was incubated for 64 hrs at 37° C. followed by the addition of 20 μL Alamar Blue and further incubation for 6 additional hrs at 37° C. prior to fluorescence measurement using a fluorescence plate reader (CytoFluor II, PerSeptive Biosystems). Alamar Blue fluorescence emission intensity was measured at 595 nm (excitation at 535 nm) and is proportional to the number of viable cells and is inversely proportional to the concentration of biologically active Emab. Activity was expressed as a percent using the following formula:
% Relative Potency=[(sample activity)/(standard activity)]×100
where the sample activity is compared to the activity expected for the total monomeric protein (or standard) expressed as a percent. The relative potencies of samples measured using this method have been found to be comparable with those obtained by the cell-based CD-22 binding assay. The accuracy of the measurement is greater than 90% and intermediate precision (CV) has been about 10%. Specificity of the bioassay has been demonstrated by showing a lack of effect toward other mAb products (Retuximab, anti-IFNγ and anti-IL-1R1). With respect to the LMW1 fragment, the results indicated a significant reduction in activity for this fragment species.

Changes in charge variance of the polypeptide particles also were determined by cation exchange chromatography (CEX). Briefly, Emab was evaluated using the cation exchange procedures known in the art. This method separated predominant C-terminal lysine isoforms based on protein surface charge differences using a linear salt gradient at pH 7 and a Dionex weak-cation exchange column (WCX-10; Sunnyvale, Calif.).

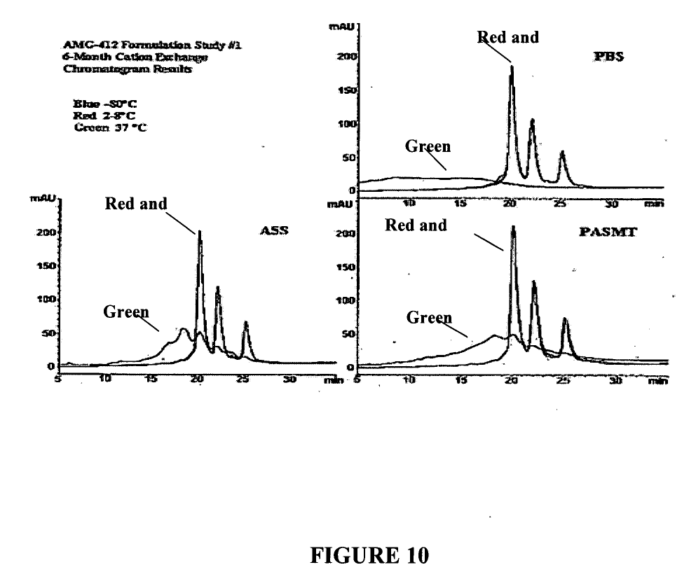

Overlayed CEX data for three different storage conditions are presented in FIG. 10 and show the changes in elution profile of three distinct species of different charge. The three charged species are assignable to predominant C-terminal lysine isoforms eluting around 20 (0K), 22 (1K) and 25 (2K) minutes. The 0K, 1K, and 2K designations refer to the number of intact heavy chain C-termini lysines. Apparent changes resulting from temperature stresses associated with the stability conditions of storage also are revealed in FIG. 10. Polypeptide stability under accelerated conditions exhibit a propensity for new peaks to form and for an overall increase in peak spreading, possibly due to deamidation and other chemical modifications altering the charge state of the polypeptide.

Comparing the overall performance of the three formulations, the results indicate that the A5S and PASMT formulations achieved better polypeptide stability than the PBS formulation. For example, the PBS formulation began to show a pre-peak shoulder of the 0K peak in the 2-8 C sample. The A5S formulation exhibited greater definition (sharper peaks) than the PASMT formulation at 37 C.

The activity of the samples also was evaluated in the bioassay described previously. The results are shown in Table 3 below. Generally, the −80 and 2-8 C samples did not exhibit any significant decline in activity. The samples maintained at 37 C showed a loss of activity with the PBS formulation exhibiting the largest activity decline. Activity in the PASMT formulation was similar the A5S formulation, with the latter exhibiting slightly better performance. Despite formation of insoluble particles in the A5S formulation stored at −80 C (see FIG. 7), there was no significant affect observed regarding a concomitant diminution in activity. This result is because the fraction of the total concentration of polypeptide that makes up the insoluble particles is insignificant. This observation is equally applicable to that of the PBS formulated product also stored at −80 C. These results indicate that removal of particles using an inline filter should have little consequence on the potency. Furthermore, these results also indicate that product formulations such as A5S or PBS are not required to be stored frozen if insoluble particle formation is to be minimized. Rather, both of these solutions would remain more stable when stored at 2-8 C. If a frozen bulk solution is required, inclusion of other excipients should be considered that confer stabilizing properties in the frozen state (i.e., sucrose, trehalose).

TABLE 3
Potency of Initial Emab Formulations
in a Bioassay after Six Months

| | | Potency Mean | CV |
|---|---|---|---|
| Formulation | Protein conc (10 mg/mL) | (n = 3) | (%) |
| A5S −80 C. | 10 | 97 | 3.8 |
| PCS −80 C. | 10 | 95 | 5.4 |
| PASMT −80 C. | 10 | 118 | 7.6 |
| A5S 2-8 C. | 10 | 99 | 10.2 |
| PBS 2-8 C. | 10 | 91 | 8.5 |
| PASMT 2-8 C. | 10 | 101 | 11.2 |
| A5S 37 C. | 10 | 75 | 14 |
| PBS 37 C. | 10 | 38 | 9.8 |
| PASMT 37 C. | 10 | 61 | 15.2 |

The extent of polypeptide oxidation and deamidation occurring in the selected formulations also was assessed. Briefly, reversed-phase HPLC/MS data of Lys-C digested samples that had been exposed to 0.7% TBHP (oxidant) at pH 5 and 7 was performed using methods well known in the art and eluting bands related to Lys-C peptides possessing oxidized methionie residues were determined. The results are summarized in Table 4 and shows that methionine residues, Met357 (heavy chain), Met427 (heavy chain), Met21 (light chain) and Met251 (light chain) had been oxidized. The percentage oxidation of each oxidized methionine also is listed in Table 4. Concerning deamidation, there was one site identified at Gln110 of the light chain that was found to undergo deamination.

TABLE 4
Oxidized Methionine Sites of AMG412
and Corresponding Percentages

| | Peptide/ | Reduced Peak | Oxidized Peak | |
|---|---|---|---|---|
| Sample | Domain | MS* Area | MS* Area | % Oxidation |
| Ref Std | Met 357 HC | 1718276484 | 6437712 | 0.373263 |
| Forced Ox | Met 357 HC | 4958587 | 601425590 | 99.18227 |
| Ref Std | Met 427 HC | 4686518657 | 372858664 | 7.369655 |
| Forced Ox | Met 427 HC | 11068860 | 1093581448 | 98.99798 |
| Ref Std | Met 21 LC | 374592803 | 250432177 | 6.071046 |
| Forced Ox | Met 21 LC | 21961419 | 1607140321 | 98.65193 |
| Ref Std | Met 251 HC | 2745339178 | 317042026 | 10.35279 |
| Forced Ox | Met 251 HC | 5111044 | 788686966 | 99.35613 |

*MS = Mass Spectrometry peak areas.

Excipient Screening and Optimization Studies

Including the preliminary excipient optimization studies described previously, a total of 68 formulations were characterized that focused on the pH region between 5 and 6. Studies under accelerated conditions were evaluated at 37 C for 4 weeks. Additional buffer compounds were considered and included histidine, acetate, and phosphate (pH 6). The sugars evaluated were sucrose, sorbitol, and glucose. Some evidence that EDTA was helpful in minimizing the fragmentation or breakdown warranted its inclusion in the formulations as described previously (at 1, 2, and 5 mM concentrations). Additionally, varying amounts of NaCl were also investigated.

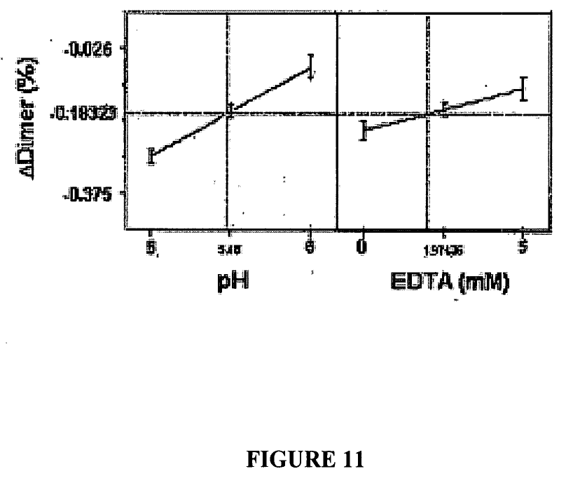

A statistical evaluation of the pH and EDTA parameters as it affects formation of the dimer peak (i.e., soluble aggregate) is shown in FIG. 11. The results further indicate that pH 5 to pH 6 minimizes dimer formation. Increasing EDTA tended to increase dimer formation.

The above results also indicated that dimer formation can be involved in the formation of insoluble particles. To address this relationship, the change in dimer formation also was determined by measuring the turbidity of the formulations. Briefly, the “St. Pauli” 845× HP Agilent UV-Visible spectrophotometer equipped with Chemstation Instrument 1 software was used for turbidity measurements. Samples were evaluated at a fixed wavelength of 400 nm (clear of protein absorbance bands) where turbidity of Emab was determined as the increase in absorbance relative to a blank. All sample measurements were taken with volumes of 500 μl in a quartz cuvette. Background compensation for the standard solutions was performed using an IN SPEC background solution prior to taking measurements with the IN SPEC standards. Five continuous measurements were obtained for each of the IN SPEC standards. A 200 μl eppendorf pipette with a costar gel loading tip attached was used to extract the solution(s) from the cuvette between subsequent measurements. Following standardization, the cuvette was removed, washed with 0.22 micron filtered water in a cuvette cleaning apparatus (single cell washer manufactured by NSG Precision Cells, Inc., Farmingdale, N.Y.) and then dried with compressed nitrogen gas. Sample measurements were made in the same identical manner with the exception that the system was blanked with the corresponding buffer in each case. The cuvette was always washed in between the different formulation runs.

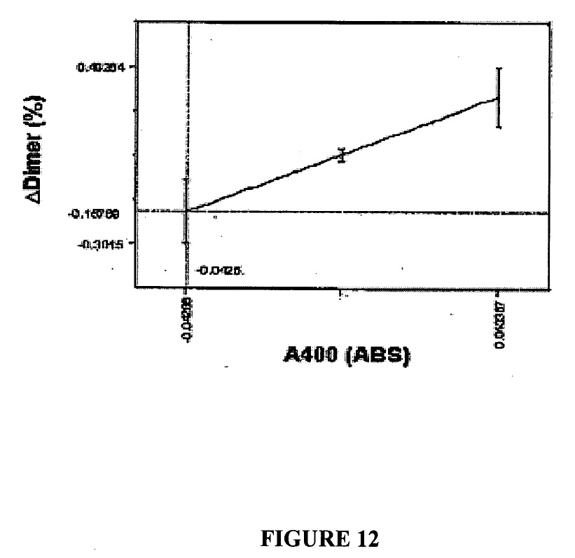

FIG. 12 shows the statistical analysis of the change in dimer concentration as measured by SEC and turbidity. The analysis indicates a weak correlation between soluble dimer and particle formation because increased dimer formation also resulted in increased turbidity. The observance of only a weak correlation can be explained by the fact that there are three distinctly different forms of dimer and that the population of any one form can be differently affected in terms of solubility.

Components and trends affecting polypeptide breakdown were analyzed as well. Several trends were observed concerning excipient properties that influence fragmentation. The influence of NaCl and EDTA on fragmentation was investigated, for example. The results indicate that NaCl did not facilitate polypeptide stability against fragmentation as measured by the LMW1 peak of the SEC. EDTA had some influence in reducing any instability imparted by NaCl. These results further indicated that EDTA can be a good excipient to include for stabilization against breakdown, while NaCl should be avoided. The correlation with the data obtained from the formulations tested was r²=0.47, however the trend appeared consistent across a broad range of formulations and differing conditions. Other studies investigating the role of divalent metal ions (i.e., Fe, Cu) further indicate that EDTA does provide some benefit in reducing the degradation reaction rate.

Similarly, examination of the influence of fragmentation with pH using trend plots as described above for NaCl and EDTA indicated that pH 6 is more preferable to pH 5. This result differs from the influence of pH on the dimer formation (see FIG. 11). Formulation conditions to minimize dimer formation can therefore require some trade-off as it pertains to the influences of pH on breakdown. Reducing both dimer formation and fragmentation together can be achieved, for example, by introducing other components such as EDTA and altering the pH to 5.5.

The stability of the formulations were additionally assessed through agitation studies. Based on the trends observed from the accelerated studies described above, four formulation sets were chosen to investigate stabilization during vigorous agitation conditions over the course of 48 hours at both room temperature (about 23 C) and refrigerated (2-8 C) conditions. The formulations included (1) A5S (10 mM acetate (pH 5), 5% sorbitol); (2) A5Su (10 mM acetate (pH 5), 5% sucrose); (3) A5.5S (10 mM acetate (pH 5.5), 5% sorbitol), and (4) A5.5Su (10 mM acetate (pH 5.5), 5% sucrose). Furthermore, to assess the influence of surfactants, either Tween-20 or Tween-80 were added in the amounts of 0, 0.005%, 0.01%, or 0.02%. In addition to the surfactant, EDTA was also evaluated at 1 mM concentration.

Briefly, the Emab material used for these studies was initially formulated in PBS. Samples were buffer exchanged into respective formulations using a Millipore lab-scale TFF system (UF/DF). Starting material was approximately 350 mL of 11.6 mg/mL. Approximately 1.5 to 2 liters of A5S (10 mM sodium acetate, pH 5, and 5% sorbitol), A5Su (10 mM sodium acetate, pH 5, and 5% sucrose), A55S (10 mM sodium acetate, pH 5.5, and 5% sorbitol) and A55Su (10 mM sodium acetate, pH 5.5, and 5% sucrose) buffers were used for the UF/DF process. The appropriate amount of Tween and EDTA were spiked into each formulation to reach the final desired concentration. 2-mL of each sterile filtered formulation was dispensed into sterile 5 cc glass vials in the sterile hood. All vials with were caped, labeled and stored at 4 C and 37 C. Samples were removed and analyzed at designated time points after vigorous shaking at 350 rpm (Signature Orbital Shaker, model DS-500) at either room temperature or at 2-8 C. Particle formation was measured by the subvisible particle method using a HIAC instrument as described previously.

The results of these agitation studies were analyzed using correlation and trend plots showing turbidity (measured using the A400) as a function of particle size distribution (based on the HIAC) considering particles≧10 micron diameters. The correlation analysis from data obtained after 48 hr. at room temperature exhibited a r² of 0.80, p<0.0001. These results also showed a reasonable correlation with the corresponding trend plot. Some outliers were identified since turbid particles also should occlude light in the HIAC instrument in a proportionate way.

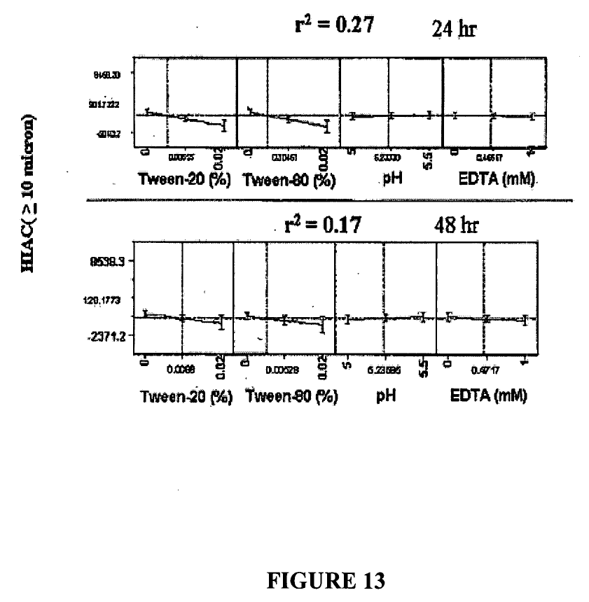

Results of trends between the HIAC particle counts≧10 μm and the influence of surfactants, pH and EDTA are shown in FIG. 13 and indicate that the pH and EDTA trends are rather flat across the ranges tested (pH 5 and 5.5, 0 and 1 mM EDTA). A weak correlation with respect to surfactants was observed. Samples containing either surfactant showed similar trends indicating that inclusion of a surfactant is beneficial for retarding insoluble particle formation. In contrast, all samples that did not contain any surfactant exhibited white masses and distinct white particles of varying size whereas in most surfactant-containing samples it was difficult to observe any sign of particulate. Therefore, inclusion of a surfactant is beneficial to reduce particulate whereas inclusion of EDTA or pH in a formulation could be varied within the tested ranges without any apparent consequence on the formation of insoluble particles.

Selection of Formulations that Augment Retention in Polypeptide Stability

The above preformulation and optimization studies lead to the selection of four candidate formulations that exhibit characteristics favoring retention in polypeptide stability. Briefly, the optimal pH range covered by the candidates is pH 5 and 5.5. Buffering capacity within this range was preliminary studied using a general acetate buffer. EDTA was shown to have some benefit with regard to retarding breakdown and was included in the selection of the candidate formulations. Based upon the agitation results it was determined that a surfactant such as Tween-20 or Tween-80 should be included to minimize particulate formation. Although sucrose and sorbitol exhibited similar properties sucrose has some can be beneficial in formulations for fructose intolerant patients. Sucrose also can be beneficial in formulations designed for long-term frozen storage because it has a higher Tg′ than sorbitol. Use of 9% sucrose also can be employed to achieve isotonicity with blood serum (i.e., about 300±/−50 mOsm/kg). Based on these results and considerations the following four formulations were determined to be formulations that facilitate retention in polypeptide stability:

1. 10 mM buffer with pKₐ 4-6 (pH 5), 5% sorbitol,0.005% Tween-20

2. 10 mM buffer with pKₐ 4-6 (pH 5), 9% sucrose, 0.005% Tween-80

3. 10 mM buffer with pKₐ 4-6 (pH 5), 9% sucrose, 0.005% Tween-80, 1 mM EDTA

4. 10 mM buffer with pKₐ 4-6 (pH 5.5), 9% sucrose, 0.005% Tween-80, 1 mM EDTA

### EXAMPLE II

### Polypeptide Stability in Propionate Buffered Aqueous Solutions

This Example shows that therapeutic polypeptides exhibit long term stability is propionic acid biopharmaceutical formulations.

To investigate the stabilizing capacity of differing buffering agents having a pKₐ in the range of 4-6, different formulations were prepared based on the candidate formulations and characteristics identified in Example I. The buffering agents that were compared included propionate, succinate and acetate. The components of each formulation is shown below in Table 5. Emab was used as the starting material and all methods used for these buffering agent comparisons were performed as described in Example I. The results of these comparisons are described further below and shown in FIGS. 14-16.

TABLE 5
Formulations for Propionate Buffer Comparisons

| Name | Buffer | Excipients | Surfactants (w/v) | pH |
|---|---|---|---|---|
| A5.0S 0.005% | 10 mM Na | 5% sorbitol | 0.005% Tween20 | 5.0 |
| Tween 20 | Acetate | | | |
| P5.0S 0.005% | 10 mM Na | 5% sorbitol | 0.005% Tween20 | 5.0 |
| Tween 20 | Propionate | | | |
| S5.0S 0.005% | 10 mM Na | 5% sorbitol | 0.005% Tween20 | 5.0 |
| Tween 20 | Succinate | | | |

A propionate buffering agent was selected for comparison with other formulations. Propionic acid or propionate exhibit optimal buffering capacity within the desired pKₐ range of 4-6. The long-term pH stability and the stability of Emab prepared in propionate formulations were determined to assess the stability of propionate buffered formulations. Polypeptide particulate or fragment formation was determined by SEC, liquid particle measurements using a HIAC instrument, osmolality and gel electrophoresis as described in Example I and according to methods well known in the art.

The pH stability of the propionate buffer (Pr5ST) compared to the acetate buffer (A5ST) set forth in Table 5 are shown in FIG. 14 for two different polypeptide concentrations (1 and 10 mg/ml). The surfactant concentration used was 0.004%. The results indicate long-term and comparable pH stability for both buffering agents for each polypeptide concentration.

Polypeptide stability formulated with a propionate buffering agent also was compared to other buffering agents under accelerated conditions. These stability assessments are shown in FIG. 15 for material eluting from SEC and are representative of results obtained for other measurements such as liquid particle formation. All three of the buffers set forth in Table 5 were compared for buffering agent performance using polypeptide concentrations at 1 and 10 mg/ml. The buffer nomenclature indicated in the FIG. 15 is: Pr—propionate; S—succinate, and A—acetate, where Na represents the salt form of the acid such as sodium propionate (e.g., NaPropionate). The results indicate that propionate buffered formulations for both polypeptide concentrations resulted in maintenance of polypeptide stability.

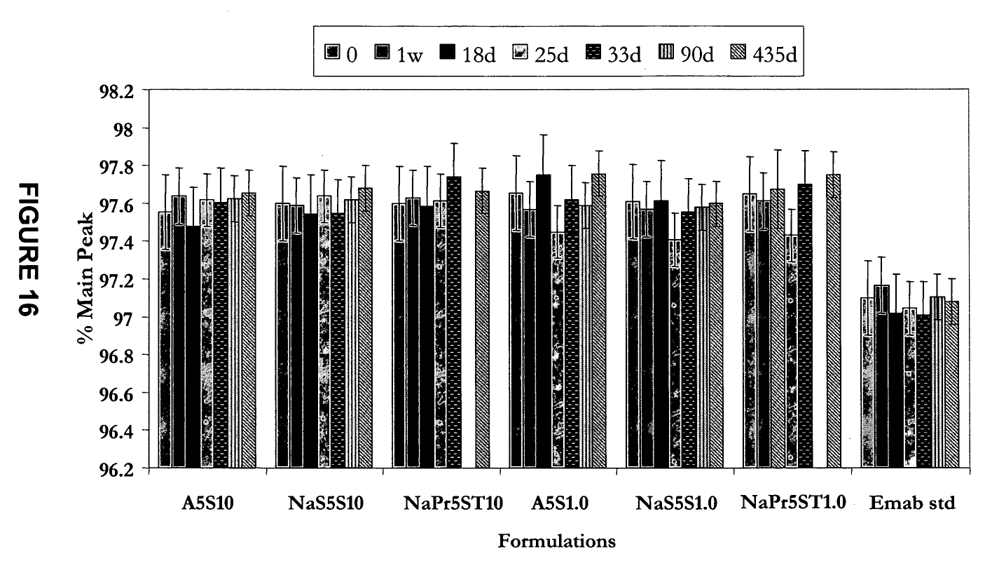

Polypeptide stability formulated with a propionate a buffering agent was further compared to other buffering agents under refrigerated conditions (4 C). These stability determinations are shown in FIG. 16 again for material eluting from SEC and are similarly representative of results obtained for other measurements. As with the formulations evaluated under accelerated conditions, all three of the buffers set forth in Table 5 were compared for polypeptide stability performance using polypeptide concentrations at 1 and 10 mg/ml. The buffer nomenclature indicated in the FIG. 16 also is the same as that described above for FIG. 15. Compared to the higher temperatures evaluate at 37 C, the results obtained at 4 C exhibit relatively minor differences between the formulations, which are consistent with the formulation characteristics described previously in Example I.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A biopharmaceutical formulation comprising an aqueous solution having a propionate buffer with a pH from about 4.0 to about 6.0, at least one excipient and an effective amount of a therapeutic polypeptide.

2. The biopharmaceutical formulation of claim 1, wherein said propionate buffer comprises sodium propionate or propionic acid.

3. The biopharmaceutical formulation of claim 1, wherein said propionate comprises a concentration selected from between about 1-50 mM, 2-30 mM, 3-20 mM, 4-10 mM and 5-8 mM.

4. The biopharmaceutical formulation of claim 1, wherein said propionate comprises a concentration of about 10 mM.

5. The biopharmaceutical formulation of claim 1, having an isotonic concentration of solutes.

6. The biopharmaceutical formulation of claim 1, where in said excipient comprises a sugar or polyols

7. The biopharmaceutical formulation of claim 6, wherein said polyols comprises a concentration selected from between about 1-10%, 2-9%, 3-8%, 4-7% and 5-6%.

8. The biopharmaceutical formulation of claim 6, wherein said polyol comprises sorbitol.

9. The biopharmaceutical formulation of claim 6, wherein said sugar comprises a concentration selected from between about 1-20% (w/v), 2-18% (w/v), 4-16% (w/v), 6-14% (w/v) and 8-12% (w/v).

10. The biopharmaceutical formulation of claim 6, wherein said sugar comprises a non-reducing sugar.

11. The biopharmaceutical formulation of claim 1, wherein said at least one excipient comprises a surfactant.

12. The biopharmaceutical formulation of claim 11, wherein said surfactant comprises a non-ionic surfactant.

13. The biopharmaceutical formulation of claim 11, wherein said non-ionic surfactant comprises a concentration selected from between about 0.001-0.10% (w/v), 0.002-0.05% (w/v), 0.003-0.01% (w/v), 0.004-0.008% (w/v) and 0.005-0.006% (w/v).

14. The biopharmaceutical formulation of claim 12, wherein said non-ionic surfactant comprises polysorbate 20.

15. The biopharmaceutical formulation of claim 1, wherein said at least one excipient further comprises two or more excipients.

16. The biopharmaceutical formulation of claim 15, wherein said two or more excipients are selected from a buffer, stabilizer, tonicity agent, bulking agent, surfactant, cryoprotectant, lyoprotectant, anti-oxidant, metal ion, chelating agent and preservative.

17. The biopharmaceutical formulation of claim 1, wherein said therapeutic polypeptide comprises an antibody, a functional fragment of an antibody, a hormone, a growth factor or a cell signaling molecule.

18. A biopharmaceutical formulation comprising an aqueous solution having between about 1-50 mM propionate with a pH from about 4.0 to about 6.0, sorbitol between about 1-20% (w/v), polysorbate 20 between about 0.001-0.10% and an effective amount of a therapeutic polypeptide.

19. The biopharmaceutical formulation of claim 18, wherein said propionate comprises a concentration of about 10 mM of sodium propionate.

20. The biopharmaceutical formulation of claim 18, wherein said pH is about 5.0.

21. The biopharmaceutical formulation of claim 18, wherein said sorbitol is about 5% (w/v).

22. The biopharmaceutical formulation of claim 18, wherein said polysorbate 20 is about 0.005% (w/v).

23. The biopharmaceutical formulation of claim 18, wherein said therapeutic polypeptide comprises an antibody, Fd, Fv, Fab, F(ab′), F(ab)₂, F(ab′)₂, single chain Fv (scFv), chimeric antibodies, diabodies, triabodies, tetrabodies, minibody, peptibody, hormone, growth factor or cell signaling molecule.

24. The biopharmaceutical formulation of claim 18, wherein said therapeutic polypeptide comprises a concentration selected from between about 10-200 mg/ml, 20-180 mg/ml, 30-160 mg/ml, 40-120 mg/ml, 50-100 mg/ml and 60-80 mg/ml.

25. A method of preparing a biopharmaceutical formulation comprising combining an aqueous solution having a propionate buffer with a pH from about 4.0 to about 6.0 and at least one excipient with an effective amount of a therapeutic polypeptide.

26. The method of claim 25, wherein said aqueous solution comprises between about 1-50 mM propionate with a pH from about 4.0 to about 6.0 and sorbitol between about 1-20% (w/v).

27. The method of claim 25, wherein said propionate comprises a concentration of about 10 mM of sodium propionate.

28. The method of claim 25, wherein said pH is about 5.0.

29. The method of claim 25, wherein said sorbitol is about 5% (w/v).

30. The method of claim 25, wherein said at least one excipient further comprises a surfactant.

31. The method of claim 30, wherein said surfactant comprises polysorbate 20 between about 0.001-0.10% (w/v).

32. The method of claim 31, wherein said polysorbate 20 is about 0.005% (w/v).

33. The method of claim 25, wherein said at least one excipient further comprises two or more excipients.

34. The method of claim 33, wherein said two or more excipients are selected from a buffer, stabilizer, tonicity agent, bulking agent, surfactant, cryoprotectant, lyoprotectant, anti-oxidant, metal ion, chelating agent and preservative.

35. The method of claim 25, wherein said therapeutic polypeptide comprises an antibody, Fd, Fv, Fab, F(ab′), F(ab)₂, F(ab′)₂, single chain Fv (scFv), chimeric antibodies, diabodies, triabodies, tetrabodies, minibody, peptibody, hormone, growth factor or cell signaling molecule.

36. The method of claim 25, wherein said therapeutic polypeptide comprises a concentration selected from between about 10-200 mg/ml, 20-180 mg/ml, 30-160 mg/ml, 40-120 mg/ml, 50-100 mg/ml and 60-80 mg/ml.

37. A container containing a biopharmaceutical formulation comprising an aqueous solution having between about 3-20 mM propionate with a pH from about 4.0 to about 6.0, sorbitol between about 1-10% (w/v), polysorbate 20 between about 0.001-0.10% (w/v) and an effective amount of a therapeutic polypeptide.
